(19) United States
(12) Patent Application Publication
(10) Pub. No.: US 2022/0168465 A1
(43) Pub. Date: Jun. 2, 2022

(54) BIODEGRADABLE NASAL SPLINT

(71) Applicant: Septum Solutions, LLC, Colleyville, TX (US)

(72) Inventors: Keith E. MATHENY, Plano, TX (US); John J. KOLENG, Austin, TX (US); Brian DORSEY, Encinitas, CA (US); Michael Scott TAYLOR, Anderson, SC (US); Brian GAERKE, Anderson, SC (US); Parimal PATEL, Anderson, SC (US); Clayton CULBREATH, Anderson, SC (US); Ryan BOREM, Anderson, SC (US)

(21) Appl. No.: 17/604,330

(22) PCT Filed: Apr. 16, 2020
(86) PCT No.: PCT/US2020/028593

§ 371 Date: Oct. 15, 2021

Related U.S. Application Data
(60) Provisional application No. 62/834,846, filed on Apr. 16, 2019.

(51) Int. Cl. A61L 27/26; A61L 27/58; A61L 27/54; A61F 5/56; A61F 5/08; A61K 31/722

(52) U.S. Cl. CPC A61L 27/26; A61L 27/58; A61L 27/54; A61F 5/56; A61F 5/08; A61L 2300/404; A61L 2300/232; A61L 2300/40; A61L 2300/406; A61L 2300/408; A61K 31/722

(57) ABSTRACT

A biodegradable nasal splint comprising a tubular component at least partially defining a hollow passageway. The tubular component may be formed from a degradable material comprising a copolymer comprising glycolide subunits, trimethyl carbonate subunits, and caprolactone subunits. The degradable material may further comprise from about 0.01% to about 30% chitosan, by weight of the degradable material. The biodegradable nasal splint may further comprise a therapeutic agent such as chitosan applied to one or more surfaces of the nasal splint Also, a biodegradable nasal splint comprising a tubular component at least partially defining a hollow passageway and formed from a degradable material. The degradable material may comprise at least about 95% chitosan, by weight of the degradable material.

### CROSS-REFERENCE TO RELATED APPLICATIONS

This application claims priority to U.S. Provisional Patent Application Ser. No. 62/834,846, entitled “Biodegradable Nasal Splint” and filed 16 Apr. 2019 and International Application No. PCT/US2020/028593, entitled “Biodegradable Nasal Splint,” and filed Apr. 16, 2020, which is incorporated herein by reference in its entirety.

### STATEMENT REGARDING FEDERALLY SPONSORED RESEARCH OR DEVELOPMENT

Not applicable.

### REFERENCE TO A MICROFICHE APPENDIX

Not applicable.

### BACKGROUND

There are many acceptable means by which therapeutic agents, be they conventional pharmaceuticals, gene therapeutics, or nanotechnologically-derived compounds, can be delivered to a location within the patient where they are required. At the same time, it may also be desirable to minimize the collateral damage or “side effects” resultant from these therapeutic agents moving elsewhere within the body where they are not therapeutically necessary, or worse, where they may be harmful or toxic. Many conventional medicines just narrowly tip the balance in favor of therapeutic benefit when weighed against the risks and side effects, either at the intended site of treatment or elsewhere within the body. For example, in the context of oncology, chemotherapeutic agents are utilized to kill malignant tumor cells at a slightly faster rate than they kill normal cells vital to the survival and health of the patient.

Conventional attempts to provide localized therapies include topical creams and ointments for dermatologic conditions, aerosolized nasal sprays for allergic rhinitis, and nebulized medications for upper and lower airway reactive inflammatory diseases such as chronic rhinosinusitis and asthma. While such therapeutics represent an improvement over non-discriminatory, systemically-administered therapeutics such as corticosteroids, which may have harsh potential side effect profiles, such therapeutics are limited in local therapeutic benefit while still resulting in local, regional, and/or distant toxicity.

Additionally, many conventional therapeutics and/or therapeutic devices are beneficial for a finite period of time, but can outlast their usefulness within the body and become potentially harmful the longer they remain within the patient.

As such, there is a need for improved means of medical therapies and/or therapeutic devices, for example, that will impart therapeutic benefit locally, while persisting for a desired, beneficial duration of time.

### SUMMARY

In some embodiments, disclosed herein is a biodegradable nasal splint comprising a tubular component at least partially defining a hollow passageway. The tubular component may be formed from a degradable material comprising a copolymer comprising glycolide subunits, trimethyl carbonate subunits, and caprolactone subunits. In some embodiments, the degradable material further comprises from about 0.01% to about 30% chitosan, by weight of the degradable material. In some embodiments, the biodegradable nasal splint further comprises a therapeutic agent such as chitosan applied to one or more surfaces of the nasal splint.

In some embodiments disclosed herein is a method comprising performing a corrective procedure with respect to a patient's nasal passage. The corrective procedure may comprise adjusting or removing at least a portion of a nasal septum of the patient or lateralizing an inferior turbinate of the patient. The method may also comprise positioning biodegradable nasal splint comprising a tubular component at least partially defining a hollow passageway within the patient's nasal passage between the nasal septum and an inferior turbinate. The tubular component may be formed from a degradable material comprising a copolymer comprising glycolide subunits, trimethyl carbonate subunits, and caprolactone subunits. In some embodiments, the degradable material further comprises from about 0.01% to about 30% chitosan, by weight of the degradable material. In some embodiments, the biodegradable nasal splint further comprises a therapeutic agent such as chitosan applied to one or more surfaces of the nasal splint

In some embodiments, disclosed herein is a biodegradable nasal splint comprising a tubular component at least partially defining a hollow passageway and formed from a degradable material. The degradable material may comprise at least about 95% chitosan, by weight of the degradable material.

In some embodiments, disclosed herein is a method comprising performing a corrective procedure with respect to a patient's ethmoid sinus. The corrective procedure may comprise removing a portion of tissue defining the ethmoid sinus. The method may also comprise positioning biodegradable nasal splint comprising a tubular component at least partially defining a hollow passageway within the patient's ethmoid sinus. The tubular component may be formed from a degradable material. The degradable material may comprise at least about 95% chitosan, by weight of the degradable material.

### BRIEF DESCRIPTION OF THE DRAWINGS

For a more complete understanding of the present disclosure and the advantages thereof, reference is now made to the following brief description, taken in connection with the accompanying drawings and detailed description:

FIG. 1 is an illustration of an embodiment of a therapeutic biodegradable support having a cylindrical configuration;

FIG. 2 is an illustration of an embodiment of a therapeutic biodegradable support having a cylindrical configuration;

FIG. 3 is an illustration of an embodiment of a therapeutic biodegradable support having a cylindrical configuration;

FIG. 4 is an illustration of an embodiment of a therapeutic biodegradable support having a cylindrical configuration;

FIG. 5 is an illustration of an embodiment of a therapeutic biodegradable support having a cylindrical configuration;

FIG. 6 is an illustration of an embodiment of a therapeutic biodegradable support having a sheet-like configuration;

FIG. 7 is an illustration of an embodiment of a therapeutic biodegradable support having a sheet-like configuration;

FIG. 8 is an illustration of an embodiment of a therapeutic biodegradable support configured as a nasal splint;

FIG. 9 is an illustration of an embodiment of a therapeutic biodegradable support configured as a nasal splint deployed within the nasal passages;

FIG. 10 is an illustration of an embodiment of a therapeutic biodegradable support configured as a nasal splint;

FIG. 11 is an illustration of an embodiment of a therapeutic biodegradable support configured as a nasal splint;

FIG. 12 is an illustration of an embodiment of a therapeutic biodegradable support configured as a nasal splint;

FIG. 13 is an illustration of an embodiment of a therapeutic biodegradable support configured as a nasal splint;

FIG. 14 is an illustration of an embodiment of a therapeutic biodegradable support configured as a nasal splint deployed within the nasal passages; and

FIG. 15 is an illustration of an embodiment of a therapeutic biodegradable support configured as a nasal splint deployed within the nasal passages.

### DETAILED DESCRIPTION OF THE EMBODIMENTS

In the drawings and description that follow, like parts are typically marked throughout the specification and drawings with the same reference numerals, respectively. The drawing figures are not necessarily to scale. Certain features of the invention may be shown exaggerated in scale or in somewhat schematic form and some details of conventional elements may not be shown in the interest of clarity and conciseness. The disclosed subject matter is susceptible to embodiments of different forms. Specific embodiments are described in detail and are shown in the drawings, with the understanding that the presently disclosed embodiments illustrated and described herein are not intended to limit the scope of the claims. It is to be fully recognized that the different teachings of the embodiments discussed herein may be employed separately or in any suitable combination to produce desired results.

Disclosed herein are embodiments of devices, apparatuses, systems, and methods associated with a therapeutic biodegradable support. In some embodiments, the therapeutic biodegradable support may be configured to apply a physical influence (e.g., a force) to a bodily tissue, such as to hold a tissue in place or to displace tissue. For example, in various embodiments, the therapeutic biodegradable support may be configured to hold open a passageway, cavity, or orifice; to constrict a passageway or cavity; to retain bodily tissue(s) together and/or in place; to inhibit constriction of a passageway or orifice; to promote constriction of a passageway; to maintain ventilation or free exchange of fluids into and/or out of one or more physiologic chambers; to promote tissue healing; or to provide hemostasis or prevent thrombosis; or combinations thereof. In some embodiments, the therapeutic biodegradable support may also be configured to provide a support at a site of deployment and/or, at least partially contemporaneously therewith, to provide a therapeutic benefit at the site of deployment or proximate thereto.

In various embodiments, the therapeutic biodegradable support may comprise any suitable form and/or configuration, for example, as may be dependent upon the intended use of the therapeutic biodegradable support. In various embodiments, the therapeutic biodegradable support may be characterized as exhibiting a desired degree of flexibility and/or rigidity and conformability and/or malleability. In some embodiments, the therapeutic biodegradable support may be characterized as mechanically or structurally active. For example, at least a portion of the therapeutic biodegradable support may be configured to apply a radially outward force (e.g., a biasing force), such as in an embodiment where the therapeutic biodegradable support is deployed within a lumen and bodily cavity. Alternatively, in some embodiments at least a portion of the therapeutic biodegradable support may be configured such that the therapeutic biodegradable support will apply a radially inward force (e.g., a biasing force), such as in an embodiment where the therapeutic biodegradable support is deployed around (e.g., at least partially encompassing) a tissue.

Additionally, in some embodiments the therapeutic biodegradable support may further comprise one or more additional components. For example, such additional components may be configured to anchor the therapeutic biodegradable support or a portion thereof, to retain the therapeutic biodegradable support in a given orientation at and/or within the site of deployment (e.g., locks, bands, straps, or ties), to retain the therapeutic biodegradable support in a given conformation, or the like. In some embodiments, the additional component may be characterized as a filament, a flange-like component, a collar-like component, an extension, or the like. In an embodiment, such one or more additional components may extend through the therapeutic biodegradable support, radially inward within the therapeutic biodegradable support, radially outward from a portion of the therapeutic biodegradable support, tangentially from the therapeutic biodegradable support, or combinations thereof. In some embodiments, the therapeutic biodegradable support may comprise a plurality of operably joined components. Alternatively, the therapeutic biodegradable support may be formed as a unitary structure.

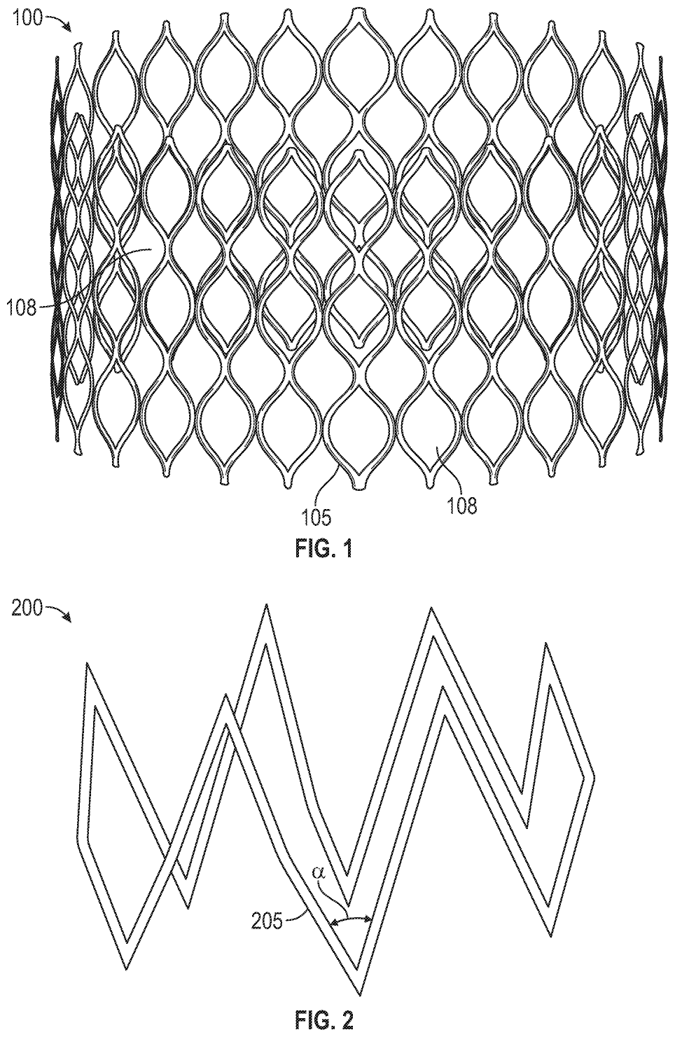

Referring to FIGS. 1 through 5, various therapeutic biodegradable support configurations are generally illustrated. In various embodiments, one or more of the general configurations disclosed with reference to FIGS. 1 through 5 may be modified, for example, for a particular purpose or use. In the embodiments of FIGS. 1 through 5, the therapeutic biodegradable support generally comprises cylinder and/or cylindrical configuration. In the embodiment of the FIG. 1, the generally cylindrical therapeutic biodegradable support is formed from a mesh-like material formed by a plurality of strands 105, for example as described herein. In alternative embodiments, a therapeutic biodegradable support like the therapeutic biodegradable support of FIG. 1 may be formed from any other suitable mesh-like material, for example, having a suitable design.

In some embodiments, the therapeutic biodegradable support may be radially expandable and/or contractible with respect to a longitudinal axis and a central passage or through bore (e.g., formed by a cylindrical wall). For example, in some embodiments the therapeutic biodegradable support may be configured so as to expand radially outward and/or to exhibit a radially outward force or bias (e.g., a radially outward mechanical activity) with respect to the longitudinal axis when uninhibited from such expansion. Alternatively, in some embodiments the therapeutic biodegradable support may be configured to contract radially inward and/or the exhibit a radially inward force or bias (e.g., a radially inward mechanical activity) with respect to the longitudinal axis when uninhibited from such contraction.

In some embodiments the therapeutic biodegradable support comprises a hollow, generally cylindrical structure having a longitudinal axis. In the embodiment of FIG. 1, such a therapeutic biodegradable support 100 that is generally hollow and cylindrical may comprise a relatively constant diameter with respect to the longitudinal axis. Alternatively, a hollow, generally cylindrical therapeutic biodegradable support may comprise a diameter that varies with respect to its length. For example, a generally cylindrical therapeutic biodegradable support may be tapered at one or both longitudinal ends, flared at one or both ends, at least partially conical (e.g., having a diameter that changes over the longitude of the therapeutic biodegradable support), or combinations thereof. The size, shape, mechanical activity, strength characteristics, and other characteristics associated with the therapeutic biodegradable support 100 may vary depending upon various considerations including, but not limited to, the intended site/locus of deployment for the therapeutic biodegradable support, the intended purpose/use for which the therapeutic biodegradable support is deployed, the intended mode of deployment, various related considerations, or combinations thereof.

In the embodiment of FIG. 2, the therapeutic biodegradable support 200, which is generally cylindrical, is formed from a plurality of struts joined at terminus or ends thereof. In various embodiments, the cylindrical therapeutic biodegradable support 200 may be formed from any suitable number of similar struts, for example, being joined at the ends thereof, alternatively, being joined at a point between the ends thereof.

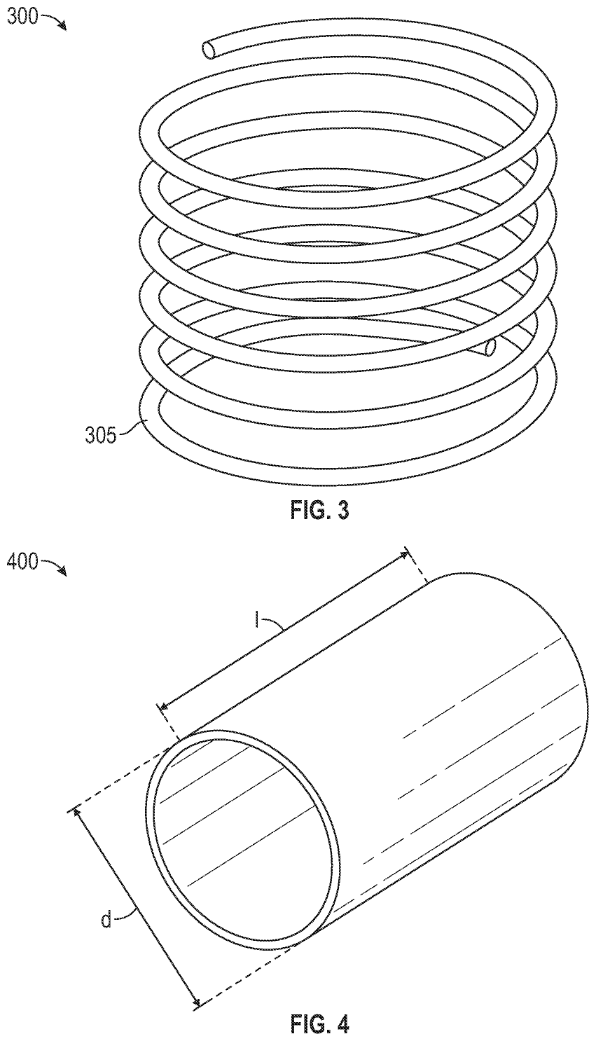

In the embodiment of FIG. 3, the therapeutic biodegradable support 300 is generally cylindrical and is formed from a helical/spiral strand. In the embodiment of FIG. 3, the therapeutic biodegradable support 300 support comprises a single, such strand. In an additional or alternative embodiment, the therapeutic biodegradable support 300 may be formed from a plurality of helical and/or spiral strands. For example, such a plurality of helical/spiral strands may coil in the same direction (e.g., a double-helix) or in opposing directions.

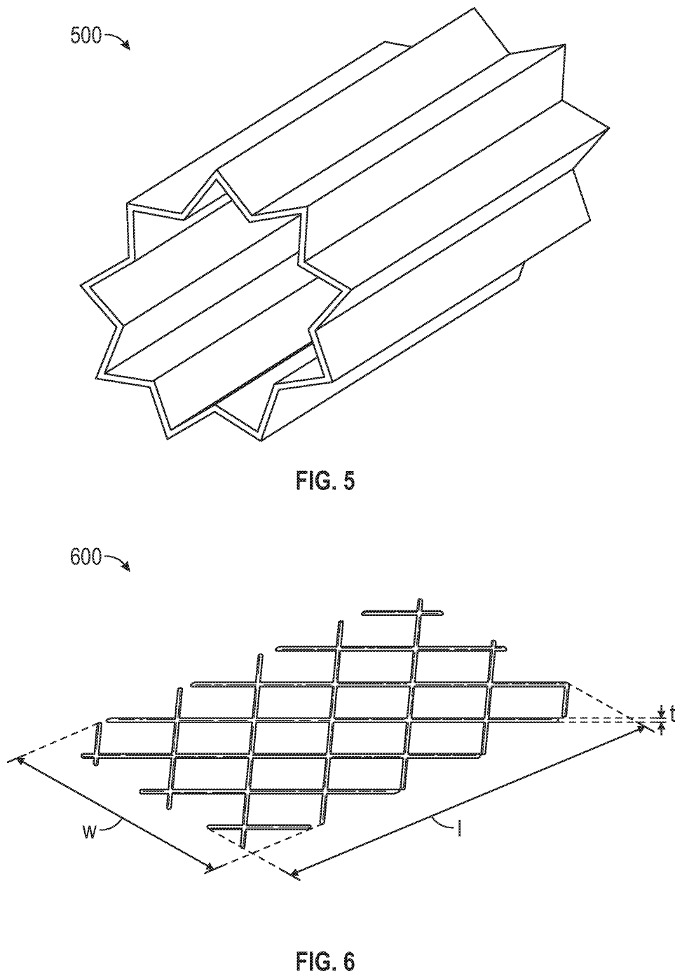

In the embodiment of FIG. 4, the therapeutic biodegradable support 400 is generally cylindrical and is formed from a sheet, a film, or a membrane. In some embodiments, the therapeutic biodegradable support 400 may be formed from a sheet, film, or membrane that has been folded and/or wrapped so as to yield the generally cylindrical configuration; alternatively, the sheet, film, or membrane may be formed having such a generally cylindrical (e.g., tubular) structure.

In the embodiment of FIG. 5, the therapeutic biodegradable support 500 is generally cylindrical and may comprise one or more pleats. In such an embodiment, the number, size, and/or separation of the pleats may be varied, for example, dependent upon the intended use of the therapeutic biodegradable support, the configuration of the therapeutic biodegradable support 500, processing parameters associated with making and/or use the therapeutic biodegradable support 500, or combinations thereof.

In some embodiments, the therapeutic biodegradable support, for example, which may be hollow and/or cylindrical, may be characterized as having a variable cross-sectional conformation. For example, the radial cross-section of the therapeutic biodegradable support may be variable such that the therapeutic biodegradable support exhibits mechanical activity. For example, in some embodiments the therapeutic biodegradable support may be radially expandable, for example, the therapeutic biodegradable support may be configured so as to expand radially outward when not retained in a relatively unexpanded conformation. In such an embodiment, the therapeutic biodegradable support may be configured to apply a radially-outward force (for example, to a lumen, passageway, or other opening) upon deployment. In an embodiment, the radially outward force applied by the therapeutic biodegradable support may be dependent upon the intended use of the therapeutic biodegradable support, the site of deployment, or other factors as may be appreciated by one of skill in the art upon viewing this disclosure. Additionally, in some embodiments the radially outward force applied by the therapeutic biodegradable support may be variable over time, for example, as may result from the degradation of a portion of the therapeutic biodegradable support. For example, in some embodiments, the radially outward force applied by the therapeutic biodegradable support may increase, alternatively, decrease, over time. In some embodiments, the therapeutic biodegradable support may comprise a nonbiodegradable component (alternatively, a component being relatively slowly degradable) that may form an outwardly-biased cylinder which is held in a partially contracted state by one or more biodegradable components, for example, such that as such as the biodegradable component degrades, the nonbiodegradable component may expand. For example, such a nonbiodegradable component (alternatively, a cylinder being relatively slowly degradable) may be configured to expand (e.g., to increase in expansion) as a tissue heals and/or as swelling subsides.

In some embodiment, a hollow, generally cylindrical therapeutic biodegradable support may be characterized as having a variable cross-sectional conformation (for example, as capable of expansion and/or contraction for placement at a site of deployment, for variability in placement, for the application of a force, or the like). In some embodiments, the therapeutic biodegradable support, or portions or components thereof may comprise a “locking” profile,” such as a ratchet, teeth, grooves, or the like, configured to lock in an expanded or contracted state upon deployment. For example, the therapeutic biodegradable support may be radially contractible, such that the therapeutic biodegradable support will contract radially inward when not retained in a relatively expanded conformation. In such an embodiment, the therapeutic biodegradable support may be configured to apply a radially-inward force (for example, to occlude or compress or narrow a lumen, passageway, or other opening) upon deployment. In some embodiments, the radially inward force applied by the therapeutic biodegradable support may be varied, for example, dependent upon the intended use of the therapeutic biodegradable support, the site of deployment, or other factors as may be appreciated by one of skill in the art upon viewing this disclosure. In some embodiments, the radially inward force applied by the therapeutic biodegradable support may be variable over time, for example, as may result from the degradation of a portion of the therapeutic biodegradable support. For example, in an embodiment, the radially inward force applied by the therapeutic biodegradable support may increase, alternatively, decrease, over time. In some embodiments, the therapeutic biodegradable support may comprise a nonbiodegradable component (alternatively, a component being relatively slowly degradable) that may form an inwardly-biased cylinder which is held in a partially expanded state by one or more biodegradable components, for example, such that as such biodegradable components degrade, the nonbiodegradable cylinder may contract (e.g., over time). For example, such a nonbiodegradable cylinder (alternatively, a cylinder being relatively slowly degradable) may be configured to contract (e.g., to narrow) over time.

In some embodiments, a hollow, generally cylindrical therapeutic biodegradable support may be suitably sized, for example, as may be dependent upon the intended use of the therapeutic biodegradable support. The dimensions of the therapeutic biodegradable support may be adapted to varying cross-sectional passageways. In some embodiments, the therapeutic biodegradable support may comprise a suitable diameter and a suitable length. For example, the outer diameter (d) may be in the range of from about 5 mm to about 3 cm, or from about 5 mm to about 1 cm, or from about 5 mm to about 20 mm. Also, the length (1) may be in a range of from about 10 mm to about 5 cm, or from about 20 mm to about 4 cm, or from about 2 cm to about 3 cm.

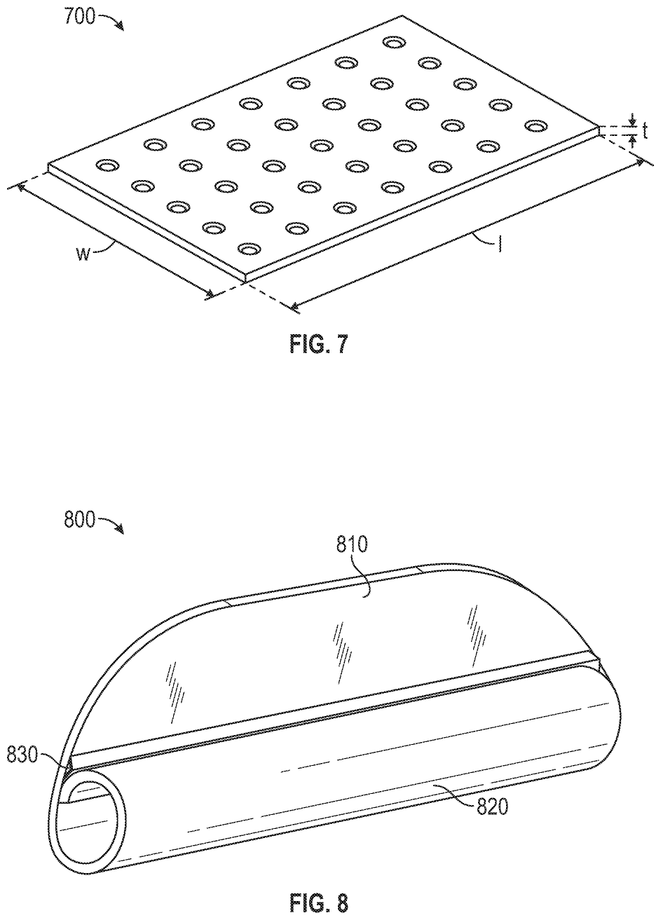

In some embodiments, the therapeutic biodegradable support generally comprises a sheet. In some embodiments, the therapeutic biodegradable support generally comprises one or more thicknesses of a suitable sheet-forming material, for example such as a mesh-like material, a film-like material, a membranous material, or combinations thereof. For example, in the embodiment of FIG. 6, the therapeutic biodegradable support 600 may comprise a sheet which may comprise one or more thicknesses of a mesh-like material, for example, as disclosed herein. Alternatively, in the embodiment of FIG. 7, the therapeutic biodegradable support 700 may comprise a sheet which may comprise one or more thicknesses of a film-like or membranous material.

In some embodiments, a therapeutic biodegradable support in the form of a sheet may be employed to form one or more suitable structures. For example, the sheet may be rolled, folded, bent, the like, or combinations thereof. In some embodiments, the sheet may be characterized as exhibiting a desired degree of conformability. For example, the sheet may be configured to be rolled, crimped, folded, stretched, compressed, pleated, or combinations thereof. Additionally or alternatively, in an embodiment the sheet may be configured so as to exhibit a tendency to remain in a particular conformation when placed in such that conformation. For example, the sheet may be characterized as malleable. Alternatively, the sheet may be configured so as to exhibit a tendency to return to a first conformation when placed in a second conformation.

In some embodiments, the sheet may be configured such that when the sheet is rolled, the sheet exhibits a tendency to unroll, for example, so as to exhibit a radially outward force. As such, such a therapeutic biodegradable support may be characterized as radially expandable, for example, the therapeutic biodegradable support may be configured so as to expand radially outward (e.g., unroll) when not retained in a rolled conformation. In some embodiments, retaining members (e.g., straps or bands) may be biodegradable such that unrolling may occur in a controlled and/or delayed fashion as directed by a given scenario. In some embodiments, the therapeutic biodegradable support may be configured to apply a radially-outward force (for example, to a lumen, passageway, or other opening) upon deployment.

Alternatively, in some embodiments, the sheet may be configured to exhibit a tendency to roll, for example, so as to exhibit a radially inward force (e.g., a centripetal force). For example, the sheet may be characterized as radially contractible, so as to contract radially inward (e.g., roll) when not retained in an unrolled and/or partially unrolled conformation. In some embodiments, biodegradable retaining members may prevent rolling, such that rolling may occur in a controlled and/or delayed fashion as directed by a given scenario. In some embodiments, the sheet may be configured to apply a radially-inward force (for example, so as to constrict a lumen, passageway, or other opening) upon deployment.

Additionally or alternatively, in some embodiments the sheet may be configured to be stretched. When the sheet is stretched, the sheet may exhibit a desired tensile strength and/or a desired bias, for example, such that the sheet exhibits a tendency to shrink in at least dimension (e.g., an elastomeric or spring-like behavior).

Additionally or alternatively, in some embodiments the sheet may be configured to be crimped or folded. For example, the sheet may be configured such that when the sheet is placed in a particular conformation (e.g., folded, crimped, squeezed, creased, or the like), the sheet exhibits a tendency to remain in that configuration. As such, in some embodiments the sheet may be formed into a suitable shape with the expectation that the sheet will remain substantially in that shape. In some embodiments, the sheet comprises a mating and/or interfacing component, for example, that forms a lock or engagement (e.g., a zip-tie-like structure).

Additionally or alternatively, two or more portions of such a sheet bonded together such that the therapeutic biodegradable support is configured to remain in a desired shaped. For example, the sheet may be bonded via the application of a suitable adhesive, by sewing (e.g., utilizing a surgical thread or suture, such as a bioabsorbable suture), by annealing (e.g., by heat and/or pressure), or by combinations thereof.

In some embodiments, the sheet may comprise any suitable shape. Examples of suitable shapes include, but are not limited to a square, a rectangle, an oval, a circle, a parallelogram, a triangle, a rhombus, a pentagon, or the like. In some embodiments, the therapeutic biodegradable support may be suitably sized. For example, in the embodiment of FIG. 7, the sheet is generally rectangular in shape and may have a length (1) in the range of from about 50 mm to about 5 cm, or from about 1 cm to about 4 cm, or from about 2 cm to about 3 cm. Also, in an embodiment the width (w) may be in a range of from about 50 mm to about 5 cm, or from about 1 cm to about 4 cm, or from about 2 cm to about 3 cm. Also, in an embodiment the sheet may have a thickness (t) in the range of from about 1 mm to about 5 mm, or from about 2 mm to about 4 mm, or from about 2 mm to about 3 mm.

Additionally, in some embodiments the sheet may be sizable. For example, the sheet may be produced in varying sizes (e.g., small, medium, or large), for example, as may be suitable dependent upon the intended use of the therapeutic biodegradable support. Additionally or alternatively, in some embodiments the sheet may be trimmable so as to achieve a desired size. For example, the sheet may be trimmed, cut, shaped, or other altered so as to yield a desired size and/or configuration, for example, utilizing conventional and readily-available tools such as scissors, knives, scalpels, or the like.

In some embodiments, the therapeutic biodegradable support may comprise a filler material. In such an embodiment the filler material may comprise a plurality of particles (e.g., a particulate material). In some embodiments, the particles may have a size ranging from about 1 μm to about 2,500 μm, or from about 10 μm to about 500 μm, or from about 25 μm to about 100 μm, or from about 50 μm to about 75 μm. In some embodiment, the particulates may be suspended (e.g., for delivery or deployment) in a carrier fluid, such as a gel, a saline solution, an aqueous solution, or combinations thereof.

In some embodiments, the therapeutic biodegradable support generally comprises and/or is characterized as a structural component. For example, the therapeutic biodegradable support may be formed from a suitable component, combinations of operably-coupled components, material(s), or combinations thereof.

In some embodiments, the therapeutic biodegradable support may be formed from one or more strands. For example, in each of the embodiments of FIGS. 1, 2, and 3, the therapeutic biodegradable supports 100, 200, and 300, respectively, comprise one or more strands, 105, 205, and 305, respectively. In some embodiments, a plurality of strands are assembled into a structure that forms all or a portion of the therapeutic biodegradable support. For example, such a plurality of strands may be assembled, connected, or interrelated to form simple or complex geometrical structures or forms. One or more of the strands may take the form of filaments, ribbons, tapes, bands, threads, or the like. The strands may comprise a suitable cross-sectional shape. For example, the strands may have a substantially flat, round, oval, square, triangular, rectangular, tear-drop-shaped, diamond, hollow, or other suitable shape or cross-section, or combinations thereof.

In some embodiments, the strand or strands may be characterized as having a suitable size, for example, thickness and/or length. For example, in some embodiments, the strands may have a thickness in the range of from about 0.5 mm to about 5 mm, or from about 1 mm to about 4 mm, or from about 2 mm to about 3 mm. Also, in some embodiments, the strands may have a length that ranges dependent upon the intended size of the therapeutic biodegradable support, the configuration of the therapeutic biodegradable support, processing parameters associated with making and/or using the therapeutic biodegradable support, or combinations thereof.

In some embodiments, the one or more strands may be operably coupled to form the therapeutic biodegradable support and/or a portion thereof. For example, in the embodiment of FIG. 1, each of the plurality of strands 105 (alternatively, a single strand which has been folded or doubled back-and-forth) comprises a generally undulating (e.g., sinusoidal in appearance) configuration, for example having a plurality of “peaks” and “troughs.” As used herein, the terms “peaks” and “troughs” are used to refer to the alternating points of inflection of the undulating strands 105, but are not to be construed as denoting any particular absolute orientation (e.g., up or down) of such strands. In the embodiment of FIG. 1, the strands 105 are positioned such that the long axis of each of the strands 105 are substantially parallel and offset such that the peak of a given strand may be joined to the trough of an adjacent strand 105. In an embodiment, the adjacent strands may be joined, connected, or interrelated (e.g., fused, adhered, or the like) by any suitable means; in an alternative embodiment, the therapeutic biodegradable support 100 may be formed such that adjacent strands comprise a unitary structure.

Alternatively, in the embodiment of FIG. 2, each of the plurality of strands 205 (alternatively, a single strand which has been folded), comprises a strut in the form of a helical segment (e.g., a portion of a helix). In the embodiment of FIG. 2, each of the strands (e.g., struts) is joined to another (e.g., an adjacent) strand (e.g., strut) at the terminal ends thereof at a suitable angle, a. Such a suitable angle, a, may be in the range of from about 10 degrees to about 140 degrees, or from about 30 degrees to about 130 degrees. As noted above, in such an embodiment, the adjacent strands may be joined (e.g., fused, adhered, or the like) by any suitable means; in an alternative embodiment, the therapeutic biodegradable support may be formed such that adjacent strands comprise a unitary structure.

In another embodiment, the therapeutic biodegradable support may comprise a single strand. For example, in the embodiment of FIG. 3, the therapeutic biodegradable support 300 is formed from a single strand 305 (e.g., a continuous strand) in the shape of a helix. In an embodiment, such a helical strand may exhibit any suitable pitch and/or helical angle. Additionally, such a helical strand may exhibit a constant pitch and/or helical angle; alternatively, such a helical strand may exhibit a variable pitch and/or helical angle.

In some embodiments, a plurality of strands may be woven and/or interlaced together. Such a plurality of strands may be woven and/or interlaced at any suitable spacing, in any suitable pattern, and/or at any suitable angle. In some embodiments, the strands may intersect at a spacing effective to yield a plurality of open-spaces 108, for example, as shown in FIG. 1, between two or more of the plurality of strands. In an embodiment, interwoven and/or interlaced strands (e.g., weft strands and warp strands) may be fused and/or adhered at the union between any two or more strands. In some embodiments, the strands may be woven into a mesh, a cloth, or other woven material.

In some embodiments, the therapeutic biodegradable support may be formed from a sheet, a film, membrane, or the like. For example, such a sheet, film, or membrane may generally be continuous, may comprise continuous portions, may be porous, or may have any other suitable configuration. In some embodiments, a sheet may be characterized as having a suitable size, for example, thickness, width and/or length. For example, in some embodiments, the sheet may have a thickness in the range of from about 0.5 mm to about 5 mm, or, from about 1 mm to about 4 mm, or, from about 2 mm to about 4 mm. Also, in an embodiment, the sheet may have a width and/or length that ranges dependent upon the intended use of the therapeutic biodegradable support, the configuration of the therapeutic biodegradable support, processing parameters associated with making and/or using the therapeutic biodegradable support, or combinations thereof. In some embodiments, the sheet may be utilized (e.g., to form the therapeutic biodegradable support) in a single layer; alternatively, in some embodiments, the sheet may be utilized (e.g., to form the therapeutic biodegradable support) in multiple layers.

In some embodiments, the therapeutic biodegradable support may comprise a molded component. For example, the therapeutic biodegradable support may be molded and/or otherwise formed, as will be disclosed herein, into any suitable shape or conformation.

In some embodiments, the therapeutic biodegradable support and/or the components and/or materials forming the therapeutic biodegradable support may be characterized as biodegradable. As used herein, the term “biodegradable” and like terms refer to a material that is configured to irreversibly be degraded or broken down into one or more constituents when deployed in a biological environment, for example, by any variety of mechanisms of degradation. For example and not intending to be limited by theory, the therapeutic biodegradable support may degrade via a surface erosion mechanism characterized by a layer by layer degradation of the therapeutic biodegradable support; additionally or alternatively, the therapeutic biodegradable support may degrade via bulk erosion characterized by erosion occurring throughout the therapeutic biodegradable support. Also not intending to be bound by theory, the therapeutic biodegradable support may degrade by any suitable mechanism, non-limiting examples of mechanisms of degradation may include hydrolysis, oxidation, aminolysis, enzymatic degradation (e.g., proteolytic degradation), physical degradation, or combinations thereof. Mechanisms of degradation may be affected through the use of external stimuli such as temperature, light, or heat. Additionally or alternatively, degradation of the therapeutic biodegradable support may occur through contact with one or more materials that facilitate chemical degradation of the therapeutic biodegradable support. For example, upon biodegradation, at least a portion of the volume of a biodegradable material (e.g., the therapeutic biodegradable support or a component thereof) may be broken down within a given duration of time upon deployment in a biological environment (for example, upon deployment so as provide contact with a bodily fluid, such as a mucosa! membrane; a bodily tissue, such as connective, muscular, nervous, and/or epithelial tissue; or combinations thereof).

Additionally, in some embodiments, the therapeutic biodegradable support and/or the components and/or materials forming the therapeutic biodegradable support may be characterized as bioabsorbable and/or resorbable. As used herein, the terms “bioabsorbable,” “resorbable,” and like terms refer to materials that, upon being degraded or broken down, the resulting constituents are assimilated, via the biological environment, for example, into the body. For example, a bioabsorbable and/or resorbable material (e.g., the therapeutic biodegradable support or a component thereof) that is degraded in a biological environment may be absorbed/resorbed by the body so as to be expelled from the body, metabolized by the body, or the otherwise dissipated.

In some embodiments, the therapeutic biodegradable support may be configured to biodegrade, for example, to a suitable extent over a suitable period of time as may be at least partially dependent upon the intended use of the therapeutic biodegradable support. For example, in some embodiments the therapeutic biodegradable support may be configured such that at least 50%, or, at least 60%, or at least 70%, or at least 80%, or at least 90%, or at least 95%, or at least 97.5%, or at least 98%, or at least 99%, or at least 99.5%, or at least 99.9% of the volume associated with the components and/or materials utilized to form the therapeutic biodegradable support may biodegrade within a suitable duration of time commencing from deployment in a biological environment. For example, in some embodiments, the materials utilized to form the therapeutic biodegradable support may biodegrade to the desired extent in a duration ranging from (A) not less than about 72 hours, or not less than about 96 hours, or not less than about 5 days, alternatively not less than about 7 days, or not less than about 14 days, or not less than about 21 days, or not less than about 28 days, or not less than about 1 month, or not less than about 2 months, or not less than about 3 months, or not less than about 4 months, or not less than about 6 months, or not less than about 8 months, or not less than about 10 months, or not less than about 12 months to (B) not more than about 7 days, or not more than about 14 days, or not more than about 21 days, or not more than about 28 days, or not more than about 1 month, or not more than about 2 months, or not more than about 3 months, or not more than about 4 months, or not more than about 6 months, or not more than about 8 months, or not more than about 10 months, or not more than about 12 months, or not more than about 15 months, or not more than about 18 months, or not more than about 24 months. Reference herein to a particular rate and/or duration of degradation associated with a therapeutic biodegradable support should not be construed as indicating that such therapeutic biodegradable support must be placed (e.g., in vivo) in order to ascertain such rate and/or duration. For example, as will be appreciated by one or skill in the art upon viewing this disclosure, a rate and/or duration of degradation may be estimated, ascertained, and/or estimated via any suitable protocol mimicking in vivo conditions at and/or proximate to the intended site of deployment, taking into consideration factors including, but not necessarily limited to, the intended site of deployment, ambient conditions at the intended site of deployment (e.g., temperature, moisture, pH, physical interaction with tissue(s), air-flow), or combinations thereof.

In some embodiments, portions or all of the therapeutic biodegradable support may be configured to degrade at a substantially constant rate. Additionally or alternatively, in some embodiments, portions or all of the therapeutic biodegradable support may be configured to degrade at a rate that varies over time. For example, in some embodiments the therapeutic biodegradable support may degrade at a generally increasing rate; alternatively, in some embodiments the therapeutic biodegradable support may degrade at a generally decreasing rate. Additionally, in some embodiments, a first component of the therapeutic biodegradable support may degrade at a first rate while a second component thereof may degrade at a second rate. For example, a first component may be entirely or substantially degraded while a second component is less than 90%, or less than 80%, or less than 70%, or less than 60%, or less than 50%, or less than 40%, or less than 30%, or less than 20% degraded. As such, the structure of the therapeutic biodegradable support may be configured to change over the course of the degradation and/or biodegradation. For example, in some embodiments, the therapeutic biodegradable support may be configured such that the rate of degradation of the therapeutic biodegradable support varies with respect to time. For example, in some embodiments the therapeutic biodegradable support may comprise a single degradable material that is degraded at a rate that varies over time. In some embodiments, multiple degradable materials (which may have differing rates of degradation) may form the therapeutic biodegradable support such that the therapeutic biodegradable support may be degraded at a rate that varies of time. For example, one or more degradable materials may form in a plurality of layers, together forming the therapeutic biodegradable support (e.g., a first layer of a first degradable material and a second layer of a second degradable material).

Additionally, in some embodiments the therapeutic biodegradable support may be configured such that the rate of degradation may be altered via the addition or subtraction of a degradation-rate modifying agent. For example, in some embodiments the therapeutic biodegradable support may be configured to exhibit a first (e.g., relatively slow) rate of degradation prior to exposure to and/or contact with a degradation accelerator and to exhibit a second (e.g., relatively fast) rate of degradation upon exposure to and/or contact with such a degradation accelerator. In such embodiments, for example, the degradation accelerator may comprise an enzymatically-active solution, an irrigating solution (e.g., a hypotonic) saline solution, radiation, such as ultra-violet (UV) radiation, sonic pulsation, the like, or combinations thereof. The addition of and/or exposure to such a degradation accelerator may have the effect of causing the therapeutic biodegradable support to rapidly degrade or “self-destruct.” Alternatively, in some embodiments the therapeutic biodegradable support may be configured to exhibit a first (e.g., relatively slow) rate of degradation while exposed to and/or in contact (e.g., either constantly or intermittently) with a degradation retarder and to exhibit a second (e.g., relatively fast) rate of degradation upon ceasing exposure to and/or contact with such a degradation retarder. For example, removal of and/or ceased exposure to such a degradation retarder may have the effect of causing the therapeutic biodegradable support to rapidly degrade or self-destruct. Additionally, in some embodiments the degradation-rate modifying agent (e.g., accelerator and/or retarder) may be included within the therapeutic biodegradable support. For example, the degradation-rate modifying agent may be encapsulated, microencapsulated, or the like, for example, such that the capsules/microcapsules may burst, rupture, or otherwise be caused to release the degradation-rate modifying agent upon a suitable stimulus.

Not intending to be bound by theory, the rate, duration, and/or extent of degradation of the therapeutic biodegradable support and/or the materials and/or components forming the therapeutic biodegradable support may be at least partially dependent upon the composition of such materials and/or components, the sizing (e.g., thickness) of such materials and/or components, environmental factors, as will be disclosed herein, or combinations thereof.

In some embodiments, the therapeutic biodegradable support may be configured such that the biodegradation of the therapeutic biodegradable support and/or the components thereof may be monitored, for example, in situ. In some embodiments the therapeutic biodegradable support may comprise a contrast media. For example, the contrast media may be incorporated within the therapeutic biodegradable support, for example, incorporated within degradable material forming the therapeutic biodegradable support. Examples of such a contrast media include, but are not limited to, a radio-contrast agent (e.g., a radio-opaque or substantially radio-opaque material, such as iodine and/or barium), a magnetic resonance imaging (MRI) contrast agent (e.g., a gadolinium-containing compound, an iron-oxide-containing compound, iron platinum particles, a manganese-containing compound), an ultrasound-enhancing material, or combinations thereof. In some embodiments, the contrast media may be detected (e.g., in vivo) and utilized to determine the amount of the therapeutic biodegradable support (or a component thereof) remaining within the body, for example, by comparison to a scale or other standard associated with the degradable material.

In some embodiments, the therapeutic biodegradable support may be formed from any suitable composition, which may be referred to as a biodegradable composition. For example, in some embodiments, the therapeutic biodegradable support may comprise, one or more degradable and/or biodegradable polymers. In some embodiments, the selection of the polymer or combination of polymers utilized may be dependent, at least in part, upon the intended use of the therapeutic biodegradable support, other components present in the therapeutic biodegradable support, the desired rate, duration, and/or extent of biodegradation, or combinations thereof. In various embodiments, a combination may take the form of a physical blend. In various embodiments, the degradable polymer may be present within the therapeutic biodegradable support in an amount of at least about 60% by weight of the therapeutic biodegradable support, or at least about 70%, or at least about 80%, or at least about 85%, or at least about 90%, or at least about 95%, or about 100% by total weight of the therapeutic biodegradable support.

In some embodiments, the degradable polymer may comprise one or more repeating subunits, for example, monomeric subunits or oligomeric subunits. Examples of subunits may include a lactide; a glycolide; a lactic acid; a glycolic acid; an ethylene glycol; a hydroxy-alkanoate; a caprolactone; an orthoester; a phosphazene; a hydroxybutyrate; a polycarbonate, such as trimethylene carbonate; an esteramide; an anhidride; a dioxanone; an alkylene alkylate; a biodegradable urethane; an amino acid; an etherester; an acetal; a cyanoacrylate; a succinimde; an anhydride such as sebacic acid, adipic acid, or terphtalic acid; an amide such as an imino carbonate or an aminio acid; a phosphate, a polyphosphonate, or a polyphosphazene; or combinations thereof, such as oligomers thereof. For example, the degradable polymer may comprise a poly(lactide); a poly(glycolide); a poly(lactide-co-glycolide); a poly(lactic acid); a poly(glycolic acid); a poly(lactic acid-co-glycolic acid); poly(lactide)/poly(ethylene glycol) copolymers; a poly(glycolide)/poly(ethylene glycol) copolymer; a polyhydroxy-alkanoate, a poly(lactide-co-glycolide)/poly(ethylene glycol) copolymer; a poly(lactic acid)/poly(ethylene glycol) copolymer; a poly(glycolic acid)/poly(ethylene glycol) copolymer; a poly(lactic acid-co-glycolic acid)/poly(ethylene glycol) copolymer; a poly(caprolactone); poly(caprolactone)/poly(ethylene glycol) copolymer; a poly(orthoester); a poly(phosphazene); a poly(hydroxybutyrate) or a copolymer including a poly(hydroxybutyrate); a poly(lactide-co-caprolactone); a polycarbonate, such as poly(trimethylene carbonate); a polyesteramide; a polyanhidride; a poly(dioxanone); a poly(alkylene alkylate); a copolymer of polyethylene glycol and a polyorthoester; a biodegradable polyurethane; a poly(amino acid); a polyetherester; a polyacetal; a polycyanoacrylate; a poly(oxyethylene)/poly(oxypropylene) copolymer, polysuccinimde; a polyanhydride such as poly(sebacic acid), poly(adipic acid), or poly(terphtalic) acid; a polyamide such as a poly(imino carbonate) or a polyaminio acid; a phosphorus-based polymer such as a polyphosphate, a polyphosphonate, or a polyphosphazene; or combinations thereof.

Biodegradable shape memory polymers, such as those commercialized by nmemoScience in Aachen, Germany, or those described in U.S. Pat. No. 5,189,110 or 5,139,832, each of which is disclosed herein in its entirety, may also be employed.

Additionally or alternatively, in some embodiments the polymer (e.g., a degradable polymer) that may be a natural polymer or a derivative there. Examples of a natural polymer and/or a derivative thereof include protein and protein-derived polymers and polysaccharides; polysaccharide-derived polymers; and lipids and lipid-derived polymers. Examples of such polymers include chitosan, chitin, collagen, albumin, gelatin, agarose, alginate, carrageenan, cellulose, hyaluronic acid, dextran, cyclodextrins, lignosulfonates, a prolamin, such as a-prolamin, zein, modified zein, casein, gelatin, gluten, serum albumin, or combinations thereof. In various embodiments, such natural polymers and/or natural polymer derivatives may be present in the form of a hydrogel or sol-gel.

For example, m some embodiments, the biodegradable composition comprises chitosan, chitin, or a combination of chitosan and chitin. Generally, chitin is a modified polysaccharide that includes beta-I, 4-glycosidically-linked N-acetyl-D-glucosamine units. Generally, chitosan is a linear polysaccharide which may be obtained by deacetylation of at least a portion of the acetyl groups present in chitin. Depending upon the degree of deacetylation, chitosan may include varying proportions of D-glucosamine units (e.g., deacetylated units) and N-acetyl-D-glucosamine units (e.g., acetylated units) which are beta-I, 4-glycosidically-linked. In some embodiments, the chitosan may be at least 50% deacetylated, referring to a polysaccharide where at least 50% of the acetyl units have been removed (e.g., where at least 50% of the N-acetyl-D-glucosamine units have been converted to D-glucosamine units). Additionally or alternatively, in some embodiments the chitosan may be at least 60% deacetylated, at least 70% deacetylated, at least 80% deacetylated, at least 85% deacetylated, at least 90% deacetylated, at least 91% deacetylated, at least 92% deacetylated, at least 93% deacetylated, at least 94% deacetylated, at least 95% deacetylated, at least 96% deacetylated, at least 97% deacetylated, at least 98% deacetylated, at least 99% deacetylated, about 100% deacetylated.

Additionally or alternatively, in some embodiments, the biodegradable composition comprises a polysaccharide, such as starch, cellulose, dextran, substituted or unsubstituted galactomannans, guar gums, high-molecular weight polysaccharides composed of mannose and galactose sugars, heteropolysaccharides obtained by the fermentation of starch-derived sugar (e.g., xanthan gum), diutan, scleroglucan, derivatives thereof, or combinations thereof. For example, in some embodiments, the biodegradable composition comprises guar or a guar derivative. Examples of guar derivatives suitable for use in the present disclosure include without limitation hydroxypropyl guar, carboxymethylhydroxypropyl guar, carboxymethyl guar, hydrophobically modified guars, guar-containing compounds, or combinations thereof. Additionally or alternatively, in some embodiments, the biodegradable composition comprises cellulose or a cellulose derivative. Examples of cellulose derivatives suitable for use in the present disclosure include without limitation cellulose ethers, ethyl cellulose, cellulose acetate, cellulose acetate propionate carboxycelluloses, carboxyalkylhydroxyethyl celluloses, hydroxyethylcellulose, hydroxypropylcellulose, carboxymethylhydroxyethylcellulose, carboxymethylcellulose, or combinations thereof.

Additionally or alternatively, in some embodiments, the biodegradable composition comprises a starch. Examples of starches suitable for use in the present disclosure include without limitation native starches, reclaimed starches, waxy starches, modified starches, pre-gelatinized starches, or combinations thereof.

In various embodiments, the degradable polymer may be made by any suitable process and/or combination of processes. For example, in some embodiments the degradable polymer may be made or otherwise formed in a process comprising melt polymerization. For example, in some embodiments where the polymer comprises one or more monomers having a “ring” structure (e.g., a poly(lactide), a poly(glycolide), a poly(lactide-co-glycolide), a poly(lactic acid), a poly(glycolic acid), and/or a poly(lactid acid-co-glycolic acid)), melt polymerization may result in the opening of such ringed structure. In some embodiments, one or more of the degradable polymers may be available with or, alternatively, without carboxylic acid end groups. In some embodiments where the end group of the degradable polymer is not a carboxylic acid (for example, where the end group comprises an ester), the resultant polymer may be referred to herein as a blocked or capped polymer. In some embodiments where the degradable polymer has a terminal carboxylic group, the resultant polymer may be referred to as an unblocked or uncapped polymer. In some embodiments, the degradable polymer may be characterized as a linear polymer, a star polymer, or combinations thereof. Additionally, in some embodiments, the degradable polymer may be characterized as a high-molecular weight polymer. For example, not intending to be bound by theory, a relatively-high-molecular weight polymer may improve the strength of the therapeutic biodegradable support and/or extend the time duration associated with degradation and/or biodegradation (e.g., bioabsorption or resorption time). Alternatively, in some embodiments a relatively low-molecular weight polymer may be utilized where degradation and/or biodegradation time is preferably less and/or where less strength is necessary.

As will be appreciated by those of skill in the art upon viewing this application, lactide monomers and/or the lactide-containing portions of a given polymer may comprise an asymmetric carbon. Suitable, racemic DL-, L-, and D-polymers, for example, as may be suitable for inclusion in the composition, are commercially available. Not intending to be bound by theory, the L-polymers may be relatively more crystalline and, as such, may degrade and/or biodegrade (e.g., adsorb, absorb, resorb, and/or dissipate) more slowly than DL-polymers. Copolymers comprising glycolide and DL-lactide or L-lactide as well as copolymers of L-lactide and DL-lactide are also commercially available. Additionally, homopolymers of lactide or glycolide are commercially available. Star polymers of lactide, glycolide, and/or lactide/glycolide copolymers are also commercially available.

In some more particular embodiments, the degradable polymer comprises lactide and/or the glycolide present within the polymer in a suitable amount. In various embodiments, the degradable polymer comprises lactide (e.g., as a monomeric unit) in an amount from about 40% to about 100% by weight, or from about 50% to about 100% by weight, or from about 60% to about 100% by weight, or from about 70% to about 100% by weight, or from about 80% to about 100% by weight, and/or glycolide (e.g., as a monomeric unit) in an amount from about 40% to about 100% by weight, or from about 50% to about 100% by weight, or from about 60% to about 100% by weight, or from about 70% to about 100% by weight, or from about 80% to about 100% by weight. In some embodiments, the degradable polymer comprises lactide and glycolide (e.g., as monomeric units) in an amount summing 100%, or about 99%, or about 95%, or about 90%. In some embodiments, the lactide and glycolide (e.g., as monomeric units) may be present in a ratio of about 85:15 lactide:glycolide, or about 75:25 lactide:glycolide, or about 65:35 lactide:glycolide, or about 50:50 lactide:glycolide, by mole.

Additionally or alternatively, in some embodiments the degradable polymer comprises chitosan and/or chitin present within the polymer in a suitable amount. In various embodiments, the degradable polymer comprises chitosan (e.g., as a monomeric unit) in an amount from about 40% to about 100% by weight, or from about 50% to about 100% by weight, or from about 60% to about 100% by weight, or from about 70% to about 100% by weight, or from about 80% to about 100% by weight, or from about 85% to about 100% by weight, or at least about 86% by weight, or at least about 87% by weight, or at least about 88% by weight, or at least about 89% by weight, or at least about 90% by weight, or at least about 91% by weight, or at least about 92% by weight, or at least about 93% by weight, or at least about 94% by weight, or at least about 95% by weight, or at least about 96% by weight, or at least about 97% by weight, and/or chitin (e.g., as a monomeric unit) in an amount from about 1% to about 50% by weight, or from about 50% to about 40% by weight, or from about 10% to about 30% by weight. In some embodiments, the degradable polymer comprises chitosan and chitin (e.g., as monomeric units) in an amount summing 100%, or about 99%, or about 95%, or about 90%, or about 85%, or about 80%. In some embodiments, the chitosan and chitin (e.g., as monomeric units) may be present in a ratio of about 85:15 chitosan:chitin, or about 75:25 chitosan:chitin, or about 65:35 chitosan:chitin, or about 50:50 chitosan:chitin, by mole.

In some embodiments, the degradable polymer may be characterized as mucoadhesive or bioadhesive. As used herein, the terms mucoadhesive and/or bioadhesive may refer to materials tending to exhibit adhesion with respect to a biological tissue, such as mucosae (e.g., one or more mucous membranes). An example of a polymer that may exhibit mucoadhesivity includes chitosan.

Additionally, in some embodiments the degradable polymer may be characterized as charged and/or ionic in character. For example, chitosan may be characterized as cationic.

Additionally, in some embodiments the degradable polymer may be characterized as therapeutic. For example, in various embodiments the degradable polymer may be characterized as having properties tending to be generally healing, curative, remedial, medicinal, restorative, or otherwise beneficial with respect to a tissue-site. Examples of degradable polymers that may be characterized as therapeutic include chitosan, chitin, hyaluronic acid, and collagen. For example, chitosan and/or chitin may be characterized as antibacterial (e.g., tending to kill bacteria), bacteriostatic (e.g., tending to stop bacterial reproduction), fungistatic (e.g., tending to stop fungal reproduction), hemostatic (e.g., tending to promote hemostasis, such as by stopping bleeding), hypoallergenic, anti-inflammatory, immunological-enhancing, biocompatible, and analgesic (e.g., tending to reduce pain.

In some embodiments, the degradable polymer of the therapeutic biodegradable support may be subjected to post-processing, for example, in order to modify one or more characteristics of the degradable polymer. For example, in some embodiments, the degradable polymer may be exposed to a radiation dosage in order to modify the degradation profile of the biodegradable support. In various embodiments, the degradable polymer may be exposed to ionizing energy such as E-beam irradiation or gamma radiation, for example, in a dosage of from about 1 kGy to about 100 kGy, or from about 30 kGy to about 60 kGy, or from about 35 kGy to about 45 kGy. Not intending to be bound by theory, exposure of the degradable polymer of the therapeutic biodegradable support may be effective to impart a desired degradation profile to the therapeutic biodegradable support, for example, such that the therapeutic biodegradable support exhibits loss of mechanical integrity within not more than about 4 weeks, more preferably within not more than about 2 weeks.

In some embodiments, the biodegradable composition may further comprise one or more suitable additives. Examples of suitable additives that may be included within the composition include, but are not limited to, preservatives, buffers, binders, disintegrants, lubricants, plasticizers, solvents, sterilizing agents, primers, and any other excipients, for example, as may be necessary and/or advantageous to impart a desired structural, functional and/or processing characteristic to the biodegradable composition and/or the therapeutic biodegradable support. For example, in an embodiment a plasticizer and/or solvent may be included in the composition, for example, for the purpose of softening (e.g., improving malleability and or conformability of) the polymer. Examples of a suitable plasticizer and/or solvent include, but are not limited to glycerol, acetone, methyl ethyl ketone, ethyl lactate, ethyl acetate, dichloromethane, ethyl acetate/alcohol blends, or combinations thereof.

Additionally or alternatively, in some embodiments an additive may be applied to (e.g., to one or more surfaces of) the biodegradable composition forming the therapeutic biodegradable support. For example, the therapeutic biodegradable support may be treated with one or more suitable additives.

For example, in some embodiments the additive may comprise a mucoadhesive. Not intending to be bound by theory, such a mucoadhesive may enhance contact between the therapeutic biodegradable support and one or more tissues at the site at which the therapeutic biodegradable support is deployed. In an embodiment, the mucoadhesive may comprise a polymer. Examples of mucoadhesive polymers include, but are not limited to, homopolymers of acrylic acid monomers such as polyacrylic acid and any of its pharmaceutically acceptable salts; copolymers of acrylic acid and methacrylic acid, styrene, or vinyl ethers; vinyl polymers such as polyhydroxyethyl acrylate, polyhydroxyethyl methacrylate, polyvinyl alcohol, and polyvinyl pyrrolidone; cellulosic derivatives such as methyl cellulose, ethyl cellulose, hydroxyethyl cellulose, hydroxypropyl cellulose, hydroxypropylmethyl cellulose, and carboxymethyl cellulose; polysaccharides such as alginic acid, sodium alginate, and tragacanth gum; collagen; gelatin; or combinations thereof.

In some embodiments, the therapeutic biodegradable support may comprise a therapeutic agent. For example, in some embodiments the biodegradable composition may comprise the therapeutic agent (e.g., as a component of the biodegradable composition forming the therapeutic biodegradable support). Additionally or alternatively, in some embodiments the therapeutic agent may be applied to (e.g., to one or more surfaces of) the biodegradable composition forming the therapeutic biodegradable support. For example, the therapeutic agent may be adherent to the therapeutic biodegradable support itself, or incorporated, or otherwise surrounded, encompassed, or sequestered within and/or by the therapeutic biodegradable support. Not intending to be bound by theory, therapeutic(s) may be incorporated into the therapeutic biodegradable support along with co-ingredients to alter, delay, hasten, or otherwise manipulate or control the rate of release and activity within the host tissue. In some embodiments, the therapeutic agent may be applied to a therapeutic biodegradable support prior to deployment, substantially contemporaneously with deployment (e.g., during the deployment procedure), following deployment, or combinations thereof. In some embodiments, the therapeutic biodegradable support may be configured to uptake one or more therapeutic agents. For example, the therapeutic biodegradable support may comprise pores, bonding agents (e.g., adhesives, molecular binders, or otherwise), thereby enabling a therapeutic agent brought into contact with the therapeutic biodegradable support to be retained.

In some embodiments, the therapeutic agent may generally comprise any suitable therapeutically-active component, any-suitable prophylactically-active component, any suitable cosmetic component, any suitable material that is safe for human use and has biological activity, or combinations thereof. In various embodiments, the therapeutic agent may comprise any ingredient suitable for the treatment, prevention, rehabilitation, therapy, alteration, minimization, amelioration, camouflage, or the like of a condition in a subject (e.g., a human “patient”), examples of which include but are not limited to chronic sinusitis, obstructive sleep apnea, postoperative inflammation/scarring/non-healing wounds, stenosis of a tubular structure due to injury or burn, middle ear inflammatory disease, Eustachian tube dysfunction, nasolacrimal duct obstruction, pharyngeal stenosis, spinal stenosis, spinal rootlet compression, esophageal stenosis, or combinations thereof.

In some embodiments, the therapeutic agent may also be the degradable polymer. For example, in some embodiments the therapeutic agent comprises chitosan and/or chitin that also forms at least some of the structure of the therapeutic biodegradable support, as disclosed herein.

Additionally or alternatively, in some embodiments the therapeutic agent may comprise any suitable pharmaceutically active component, any suitable active pharmaceutical ingredient (API). Examples of an API that may be included within the therapeutic biodegradable support include, but are not limited to, anticholinergic agents, anti-infective agents (e.g., an antibiotic, such as antibacterial agents, antifungal agents, antiparasitic agents, antiviral agents, antiseptics or combinations thereof), anti-inflammatory agents (such as steroidal and/or nonsteroidal anti-inflammatory agents), antiscarring or antiproliferative agents, chemotherapeutic/antineoplastic agents, cytokines, decongestants, extracellular signaling/intracellular signaling molecules, healing-promotion agents and vitamins, hemostatic agents, hormones, hyperosmolar agents, immunoglobulins, immunomodulators, immunosuppressive agents, leukotriene modifiers, mast cell stabilizers, mitotic inhibitors, mucolytics, muscle relaxants, narcotic analgesics, non-narcotic analgesics, nucleic acids, other peptides, other proteins including potential allergens for immunotherapy (e.g., pollen), proton-pump inhibitors, sclerosing agents, tyrosine kinase inhibitors, vasoactive agents or combinations thereof. Additionally, anti-sense nucleic acid oligomers or other direct transactivation and/or transrepression modifiers of mRNA expression, transcription, and protein production may also be used.

Examples of suitable anticholinergic agents may generally include antimuscarinic agents, ganglionic blockers, neuromuscular blockers, or combinations thereof. Examples of anticholinergic agents include, but are not limited to, ipratroprium bromide, dicycloverine, atropine, benztropine, ipratropium, oxitropium, tiotropium, glycopyrrolate, oxy butnin, tolterodine, diphenhydramine, dimenhydrinate, or combinations thereof.

Examples of suitable antihistamines may generally include Hi-receptor antagonists, H2-receptor antagonists, H3-receptor antagonists, H4-receptor antagonists, histidine decarboxylase inhibitors, mast cell stabilizers, or combinations thereof. Examples of suitable Hi-receptor antagonists include, but are not limited to, azelastine, diphenhydramine, chlorpheniramine, meclozine, promethazine, loratadine, desloratadine, fexofenadine, cetirizine, levocetirizine, olopatadine, brompheniramine, buclizine, bromodiphenhydramine, carbinoxamine, chlorpromazine, cyclizine, chlorpheniramine, clemastine, cyproheptadine, dexbromheniramine, deschlorpheniramine, dexchlorpheniramine, dimenhydrinate, dimetindene, doxylamine, ebastine, embramine, orphenadrine, phenindamine, pheniramine, phenyltoloxamine, pyrilamine, quetiapine, rupatadine, tripelennamine, and trirolidine. Examples of suitable H2-receptor antagonists include, but are not limited to, cimetidine, famotidine, lafutidine, nizatidine, ranitidine, and roxatidine. Examples of suitable H3-receptor antagonists include, but are not limited to, ciproxifan, clobenpropit, conessine, and thioperamide. Examples of suitable H4-receptor antagonists include, but are not limited to, thioperamide. Examples of suitable histidine decarboxylase inhibitors include tritiqualine and catechin. Examples of suitable mast cell stabilizers include cromoglicate, medocromil, cromolyn sodium, and 2 adrenergic agonists.

Examples of suitable antibacterial agents include, but are not limited to, aminoglycosides, amphenicols, ansamycins, -lactams, lincosamides, macrolides, nitrofurans, quinolones (e.g., levofloxacin), sulfonamides, sulfones, tetracyclines, vancomycin, and any of their derivatives, or combinations thereof.

Examples of suitable -lactams include, but are not limited to, carbacephems, carbapenems, cephalosporins, cephamycins, monobactams, oxacephems, penicillins, and any of their derivatives, or combinations thereof.

Examples of suitable penicillins include, but are not limited to, amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin G benzathine, penicillin G benzhydrylamine, penicillin G calcium, penicillin G hydrabamine, penicillin G potassium, penicillin G procaine, penicillin N, penicillin 0, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, and ticarcillin. In an embodiment, penicillins may be combined with clavulanic acid. An example of a suitable combination of a penicillin and clavulanic acid is Augmentin™ (amoxicillin and clavulanic acid).

Examples of suitable antifungal agents include, but are not limited to, allylamines, imidazoles, polyenes, thiocarbamates, triazoles, and any suitable derivatives thereof.

Examples of suitable antiparasitic agents include atovaquone, clindamycin, dapsone, iodoquinol, metronidazole, pentamidine, primaquine, pyrimethamine, sulfadiazine, trimethoprim/sulfamethoxazole, trimetrexate, or combinations thereof.

Examples of suitable antiviral agents include, but are not limited to, acyclovir, famciclovir, valacyclovir, edoxudine, ganciclovir, foscamet, cidovir (vistide), vitrasert, formivirsen, HPMPA (9-(3-hydroxy-2-phosphonomethoxypropyl)adenine), PMEA (9-(2-phosphonomethoxyethyl)adenine), HP MPG (9-(3-Hydroxy-2-(Phosphonomet-hoxy)propyl)guanine), PMEG (9-[2-(phosphonomethoxy)ethyl]guanine), HP MPC (1-(2-phosphonomethoxy-3-hydroxypropyl)-cytosine), ribavirin, EICAR (5-ethynyl-1-beta-D-ribofuranosylimidazole-4-carboxamine), pyrazofurin (3-[beta-D-ribofuranosyl]-4-hydroxypyrazole-5-carboxamine), 3-Deazaguanine, GR-92938X (1-beta-D-ribofuranosylpyrazole-3,4-dicarboxami-de), LY253963 (1,3,4-thiadiazol-2-yl-cyanamide), RD3-0028 (1,4-dihydro-2,3-Benzodithiin), CL387626 (4,4′-bis[4,6-d]3-aminophenyl-N,N-bis(2-carbamoylethyl)-sulfonilimino]-1-3,5-triazin-2-ylamino-biphenyl-2-,2′-disulfonic acid disodium salt), BABIM (Bis[5-Amidino-2-benzimidazoly-1]-methane), NIH351, and combinations thereof.

In some embodiments, an anti-inflammatory agent may comprise a steroidal anti-inflammatory agent (e.g., a corticosteroid). Examples of suitable steroidal anti-inflammatory agents include 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, any of their derivatives, or combinations thereof.

Additionally or alternatively, in some embodiments the anti-inflammatory agent may comprise a nonsteroidal anti-inflammatory agent. Examples of suitable nonsteroidal anti-inflammatory agents include, but are not limited to, COX inhibitors (COX-1 or COX nonspecific inhibitors) (e.g., salicylic acid derivatives, aspirin, sodium salicylate, choline magnesium trisalicylate, salsalate, diflunisal, sulfasalazine and olsalazine; para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin; anthranilic acids (fenamates) such as mefenamic acid and meloxicam; enolic acids such as the oxicams (piroxicam, meloxicam) and alkanones such as nabumetone); selective COX-2 inhibitors (e.g., diaryl-substituted furanones such as rofecoxib; diaryl-substituted pyrazoles such as celecoxib; indole acetic acids such as etodolac and sulfonanilides such as nimesulide); or combinations thereof.

Examples of anti-scarring agents include, but are not limited to, silicon and vitamin E and derivatives thereof, or combinations thereof.

Examples of antiseptics include, but are not limited to chlorhexadine, benzalkonium chloride, octenidine, boric acid, cetyl trimethylammonium bromide, cetylpyridium chloride, benzethonium chloride, povidone, betadine, polyhexanide, iodine and derivatives thereof, or combinations thereof.

Examples of suitable chemotherapeutic/antineoplastic agents include, but are not limited to antitumor agents (e.g., cancer chemotherapeutic agents, biological response modifiers, vascularization inhibitors, hormone receptor blockers, cryotherapeutic agents or other agents that destroy or inhibit neoplasia or tumorigenesis) such as alkylating agents or other agents which directly kill cancer cells by attacking their DNA (e.g., cyclophosphamide, isophosphamide), nitrosoureas or other agents which kill cancer cells by inhibiting changes necessary for cellular DNA repair (e.g., carmustine (BCNU) and lomustine (CCNU)), antimetabolites and other agents that block cancer cell growth by interfering with certain cell functions, usually DNA synthesis (e.g., 6 mercaptopurine and 5-fluorouracil (5FU), antitumor antibiotics and other compounds that act by binding or intercalating DNA and preventing RNA synthesis (e.g., doxorubicin, daunorubicin, epirubicin, idarubicin, mitomycin-C and bleomycin) plant (vinca) alkaloids and other anti-tumor agents derived from plants (e.g., vincristine and vinblastine), steroid hormones, hormone inhibitors, hormone receptor antagonists and other agents which affect the growth of hormone-responsive cancers (e.g., tamoxifen, herceptin, aromatase inhibitors such as aminoglutethamide and formestane, trriazole inhibitors such as letrozole and anastrazole, steroidal inhibitors such as exemestane), antiangiogenic proteins, small molecules, gene therapies and/or other agents that inhibit angiogenesis or vascularization of tumors (e.g., meth-1, meth-2, thalidomide), bevacizumab (Avastin), squalamine, endostatin, angiostatin, Angiozyme, AE-941 (Neovastat), CC-5013 (Revimid), medi-522 (Vitaxin), 2-methoxyestradiol (2ME2, Panzem), carboxyamidotriazole (CAI), combretastatin A4 prodrug (CA4P), SU6668, SU1 1248, BMS-275291, COL-3, EMD 121974, IMC-1C11, IM862, TNP-470, celecoxib (Celebrex), rofecoxib (Vioxx), interferon alpha, interleukin-12 (IL-12) or any of the compounds identified in Science Vol. 289, Pages 1197-1201 (Aug. 17, 2000), which is incorporated herein by reference in its entirety, biological response modifiers (e.g., interferon, bacillus calmette-guerin (BCG), monoclonal antibodies, interluken 2, granulocyte colony stimulating factor (GCSF), etc.), PGDF receptor antagonists, herceptin, asparagmase, busulphan, carboplatin, cisplatin, carmustine, cchlorambucil, cytarabine, dacarbazine, etoposide, flucarbazine, flurouracil, gemcitabine, hydroxyurea, ifosphamide, irinotecan, lomustine, melphalan, mercaptopurine, methotrexate, thioguanine, thiotepa, tomudex, topotecan, treosulfan, vinblastine, vincristine, mitoazitrone, oxaliplatin, procarbazine, streptocin, taxol or paclitaxel, taxotere, analogs/congeners, derivatives of such compounds, or combinations thereof.

Examples of cytokines include, but are not limited to interferon (such as, but not limited to type I, type II, and type III interferons) interleukins, or combinations thereof.

Examples of proton-pump inhibitors include, but are not limited, dexlansoprazole, esomeprazole, lansoprazole, omeprazole, pantoprazole, rabeprazole, or combinations thereof.

Examples of decongestants include, but are not limited to, epinephrine, pseudoephedrine, oxymetazoline, phenylephrine, tetrahydrozolidine, xylometazoline, or combinations thereof.

Examples of healing-promotion agents and vitamins include, but are not limited to, retinoic acid, vitamin A, vitamin D, vitamin E, vitamin K, and derivatives thereof, or combinations thereof.

Examples of hemostatic agents include, but are not limited to, aminocaproic acid, desmopressin, or combinations thereof.

Examples of potential hormones include, but are not limited to, androgens (such as aldosterone, testosterone, or dehydroepiandrosterone), anti-androgens, estrogens, anti-estrogens, progesterone, estradiol, GnRH analogs, steroids, sterols, growth hormones, anti-diuretic hormone, melatonin, serotonin, thyroxine, triiodothyronine, calcitonin, thyroid-stimulating hormone (TSH), parathyroid hormone, glucagon, epinephrine, norepinephrine, dopamine, oxytocin, insulin, insulin-like growth factor, and analogs thereof, or combinations thereof.

In an embodiment where it is desirable to remove water from a tissue (e.g., to remove fluid from polyps or edematous tissue) a hyperosmolar agent may be employed. Examples of suitable hyperosmolar agents include, but are not limited to, furosemide, sodium chloride gel, or other salt preparations that draw water from tissue or substances that directly or indirectly change the osmolar content of the mucous layer.

In an embodiment, immunomodulators may generally comprise immunosuppressants, immunostimulants, tolerogens, or combinations thereof. Examples of immunomodulators include imiquimod, cyclosporine, tacrolimus, azathioprine, cyclophosphamide, methotrexate, chlorambucil, mycophenolate mofetil (MMF), prednisolone, levamisole, thalidomide, or combinations thereof.

Examples of leukotriene modifiers include, but are not limited to, montelukast.

Examples of mitotic inhibitors include, but are not limited to, mitomycin-C, taxanes, topoisomerase inhibitors, vinca alkaloids, or combinations thereof.

Examples of muscle relaxants include cyclobenzaprine, dantrolene, metaxalone, tizanidine, or combinations thereof.

Examples of mucolytics include, but are not limited to, acetylcysteine, domase alpha, guafenesin, or combinations thereof.

Examples of narcotic analgesics include, but are not limited to, codeine, fentanyl, hydrocodone, hydromorphone, meperidine, methadone, morphine, oxycodone, oxymorphone, or combinations thereof.

Examples of non-narcotic analgesics include, but are not limited to, acetaminophen, aspirin, celecoxib, diclofenac, ibuprofen, indomethacin, ketorolac, misoprostol, meloxicam, naproxen sodium, or combinations thereof.

Examples of vasoactive agents include, but are not limited to, nitrates, vasoconstrictors, vasodilators, or combinations thereof.

In various embodiments, the API may be present in any suitable form. For example, in some embodiments, the API may comprise a pharmaceutically acceptable salt, a suitable prodrug, a suitable solvate (e.g., a hydrous crystalline form an anhydrous crystalline form), or derivatives thereof, or combinations thereof.

As used herein, the term “pharmaceutically acceptable salt” refers to a salt of an active compound which is prepared with relatively non-toxic acids. Acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, toluenesulfonic (including p-toluenesulfonic, m-toluenesulfonic, and o-toluenesulfonic), citric, tartaric, methanesulfonic, and the like. Also included are salts of amino acids such as arginate and the like, and salts of organic acids like glucuronic or galactunoric acids and the like (see, for example, Berge et al. J. Pharm. Sci. 66:1-19 (1977, which is incorporated herein by reference in its entirety)).

As used herein, the term “prodrug,” refers to a compound that is a drug precursor which, upon administration to a subject, undergoes chemical conversion by metabolic or chemical processes to yield a desired compound (e.g., an API) or a salt and/or solvate thereof (e.g., a prodrug on being brought to the physiological pH or through enzyme action is converted to the desired drug form). A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated in their entirety herein by reference.

As used herein, the term “solvate” refers to a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is water.

In some embodiments, the API may be present in the therapeutic biodegradable support in an amount of from about 0.01% to about 95%, from about 0.01% to about 90%, from about 0.01% to about 80%, from about 0.01% to about 70%, from about 0.01% to about 60%, from about 0.01% to about 50%, from about 0.01% to about 40%, from about 0.01% to about 30%, from about 0.01% to about 20%, from about 0.01% to about 10%, from about 0.01% to about 5%, from about 0.01% to about 1%, or from about 0.01% to about 0.25% by the total weight of the therapeutic biodegradable support. Not intending to be bound by theory, the amount of API included within the therapeutic biodegradable support may depend upon one or more factors including, but not limited to, the particular ingredient incorporated, any other APis that are also incorporated, the condition or conditions being treated, the progression of the condition or conditions being treated, the age of the patient, the gender of the patient, the size of the patient, the intended site of deployment, the severity of clinical symptoms, the desired or approved dosage regimen, and combinations of these factors.

In some embodiments, the therapeutic biodegradable support may be configured such that the API is eluted (e.g., released) from the therapeutic biodegradable support at a suitable rate. For example, the therapeutic biodegradable support may be configured to elute the API at a rate, per day, in the range from (A) at least about 0.01 μg, or at least about 0.1 μg, or at least about 1 μg, or at least about 5 μg, or at least about 10 μg, or at least about 25 μg, alternatively at least about 50 μg, or at least about 75 μg, or at least about 100 μg, or at least about 150 μg, or at least about 200 μg, to (B) at most about 10 μg, or at most about 25 μg, or at most about 30 μg alternatively, at most about 40 μg, or at most about 50 μg, or at most about 75 μg, or at most about 100 μg, or at most about 200 μg, or at most about 300 μg, or at most to about 400 μg, or at most to about 500 μg, or at most to about 600 μg, or at most to about 700 μg, or at most to about 800 μg, or at most to about 900 μg, or at most to about 1,000 μg, or at most to about 1,250 μg, or at most to about 1,500 μg, or at most to about 2,000 μg. Not intending to be bound by theory, the rate of elution of the API may depend upon one or more factors, examples of which include, but are not limited to those factors set forth above.

In some embodiments, the API may be eluted from the therapeutic biodegradable support over a suitable period of time, for example, as may be at least partially dependent upon the intended use of the therapeutic biodegradable support. For example, in some embodiments the API may be eluted from the therapeutic biodegradable support such that at least 50%, or at least 60%, or at least 70%, or at least 80%, or at least 90%, or at least 95%, or at least 97.5%, or at least 98%, or at least 99%, or at least 99.5%, or at least 99.9% of the API associated with the therapeutic biodegradable support will be eluted therefrom within a suitable duration of time. For example, in an embodiment, the API may be eluted to the desired extent in a duration ranging from (A) not less than about 72 hours, or not less than about 96 hours, or not less than about 5 days, or not less than about 7 days, or not less than about 14 days, or not less than about 21 days, or not less than about 28 days, or not less than about 1 month, or not less than about 2 months, or not less than about 3 months, or not less than about 4 months, or not less than about 6 months, or not less than about 8 months, or not less than about 10 months, or not less than about 12 months to (B) not more than about 7 days, or not more than about 14 days, or not more than about 21 days, or not more than about 28 days, or not more than about 1 month, or not more than about 2 months, or not more than about 3 months, or not more than about 4 months, or not more than about 6 months, or not more than about 8 months, or not more than about 10 months, or not more than about 12 months, or not more than about 15 months, or not more than about 18 months, or not more than about 24 months.

In some embodiments, the duration over which the API is eluted may be less than, substantially the same as, or the same as the duration over which the therapeutic biodegradable support degrades. For example, in some embodiments, the elution of the API may be contemporaneous with the degradation and/or biodegradation of the therapeutic biodegradable support. In some alternative embodiments, the elution of the API may be partially contemporaneous with the degradation and/or biodegradation of the therapeutic biodegradable support. For example, the elution of the API may begin substantially contemporaneously with biodegradation of the therapeutic biodegradable support or may begin after biodegradation of the therapeutic biodegradable support has begun. Additionally or alternatively, the elution of the API may end substantially contemporaneously with biodegradation of the therapeutic biodegradable support or may end before biodegradation of the therapeutic biodegradable support has ended.

In various embodiments, the API may be added to the therapeutic biodegradable support in any suitable manner, for example, as will allow the API to be eluted from or by the therapeutic biodegradable support. For example, in some embodiments the active agent may be coated onto a surface of the therapeutic biodegradable support or a portion thereof. For example, the API may be dissolved or suspended in a solution, which may be applied to one or more surfaces of the therapeutic biodegradable support (e.g., by spraying, dipping, bathing, adsorption, absorption, or the like). Additionally or alternatively, the API may be powder coated onto one or more surfaces of the therapeutic biodegradable support and adhered thereto, for example, by heating the therapeutic biodegradable support, softening the therapeutic biodegradable support with a plasticizer, or by any suitable process.

Additionally or alternatively, in some embodiments the API may be incorporated into the therapeutic biodegradable support or a portion thereof. For example, in some embodiments the API may be incorporated throughout all portions of the device or in particular portions of the device. For example, the API may be incorporated into the biodegradable composition (e.g., the degradable polymer) utilized to form the therapeutic biodegradable support. In some embodiments, the API may be dispersed within the polymer matrix such that, as the degradable polymer degrades, the incorporated API is released. Additionally or alternatively, the therapeutic biodegradable support may comprise a plurality of pores retaining API, so as to act as a mechanical and/or physical barrier to release of the API, for example, such that the API may be released via erosion, diffusion, or combinations thereof, of the therapeutic biodegradable support. Additionally or alternatively, the API may be encapsulated and/or microencapsulated and the encapsulated/microencapsulated API may be added to the therapeutic biodegradable support (e.g., impregnated into the surface of the therapeutic biodegradable support) or incorporated within the biodegradable composition that forms the therapeutic biodegradable support.

In some embodiments, the therapeutic biodegradable support may be configured such that the one or more APis may be eluted differently with respect to time. For example, in some embodiments, a single API may be added to the therapeutic biodegradable support such that the API is eluted at a rate that varies over time. In some embodiments, multiple APis may be added to the therapeutic biodegradable support (e.g., the therapeutic biodegradable support) such that the multiple APis are eluted at independent rates. For example, one or more APis may be added to the therapeutic biodegradable support in a plurality of layers, within pockets, in reservoirs, encapsulated in microspheres which may allow the release of the API at different rates. Additionally, in some embodiments a portion of the API may be quick-release (e.g., configured for immediate or substantially-immediate release from the therapeutic biodegradable support) and another portion of the API may be extended release (e.g., configured for release, from the same or a difference portion of the therapeutic biodegradable support, over a relatively long duration of time).

In some embodiments, the therapeutic biodegradable support may be characterized as exhibiting suitable mechanical strength in order to achieve an intended result when deployed, for example, to support tissue regrowth; to maintain damaged biological tissue in a particular orientation or placement; to correct deformed biological tissue; to maintain the patency of a passageway, cavity, or orifice; to constrict a passageway, cavity, or orifice; to inhibit constriction of a passageway, cavity, or orifice; to promote constriction of a passageway, cavity, or orifice; to maintain ventilation or free exchange of fluids into and/or out of a physiologic chamber; to physically impede access to a physiologic chamber; or combinations thereof.

For example, in some embodiments the therapeutic biodegradable support may be characterized as exhibiting resistance to compression and/or elastic recovery

In some embodiments, the therapeutic biodegradable support may be made according to any suitable methodology. In some embodiments, the therapeutic biodegradable support may be fabricated via an additive manufacturing process, such as by 3-D printing or injection-molding. Generally, in such additive manufacturing processes, layers of biodegradable composition are selectively joined or fused together in a predetermined pattern to yield the therapeutic biodegradable.

Additionally or alternatively, in some embodiments the therapeutic biodegradable support may be fabricated via a subtractive manufacturing process, such as CNC milling (e.g., CNC routing) or laser cutting. Generally, in such subtractive manufacturing processes, predetermined portions of material are removed from a blank formed from the biodegradable composition to yield the therapeutic biodegradable support.

In some embodiments, the therapeutic agent may be included within the biodegradable composition (e.g., within the composition used to in the 3-D printing or injection-molding process or to form the blank used to mill the therapeutic biodegradable support). Additionally or alternatively, in some embodiments the therapeutic agent may be added to the therapeutic biodegradable support after the therapeutic biodegradable support has been formed. For example, the therapeutic agent may be coated onto the surface of the therapeutic biodegradable support or a portion thereof. For example, the therapeutic agent may be dissolved or suspended in a solution, which may be applied to one or more surfaces of the therapeutic biodegradable support, such as by spraying, dipping, bathing, adsorption, absorption, or the like. Alternatively, the therapeutic support may be coated onto one or more surfaces of the therapeutic biodegradable support as a powder and adhered thereto, for example, by heating the therapeutic biodegradable support and/or softening the therapeutic biodegradable support with a plasticizer.

Additionally, in some embodiments the therapeutic biodegradable support may be irradiated, for example, via ionizing energy (e.g. E-beam irradiation or gamma radiation) from about 1 kGy to about 100 kGy, or from about 30 kGy to 60 kGy, or from about 35 kGy to about 45 kGy. In some embodiments, the therapeutic biodegradable support may be irradiated prior to inclusion of any therapeutic agent. Additionally or alternatively, in some embodiments the therapeutic biodegradable support may be irradiated after inclusion of any therapeutic agent. Irradiation may also occur in the final package.

In some embodiments, the therapeutic biodegradable support may be utilized or used in the treatment of a patient. In some embodiments, a therapeutic biodegradable support is configured for use in treating a patient in need thereof. In some embodiments, the patient may be generally characterized as experiencing a dysfunction, undesirable medical condition, disorder, or disease state. The dysfunction, undesirable medical condition, disorder, or disease state will be collectively referred to hereinafter as an “undesirable condition.” For example, the undesirable condition may include conditions such as “genetic diseases” which refer to conditions attributable to one or more gene defects. An “undesirable condition” may also include “acquired pathologies” which refer to pathological conditions that are not attributable to inborn defects, cancers, diseases, and the like. Examples of “acquired pathologies” include, but are not limited to, infectious diseases, post-operative states, or traumatic defects. Additionally, an “undesirable condition” may include structural or functional conditions, for example, that develop throughout life, such as upper airway collapse during inspiration (e.g., as seen in obstructive sleep apnea), chronic Eustachian tube dysfunction, chronic rhinosinusitis, vertebral disk disease, presbyphonia, natural cosmetic aging, peripheral vascular disease, fertility beyond the timeframe of desired reproduction, Type II diabetes mellitus, morbid obesity, or the like. In an embodiment, the undesirable condition may comprise one or more of the conditions or states disclosed herein or any other condition or state as may be appreciated by one skill in the art upon viewing this disclosure.

Herein “treatment” refers to an intervention performed with the intention of preventing the development or altering the pathology of such an undesirable condition. Accordingly “treating” refers both to therapeutic treatments and to prophylactic measures. In some embodiments, administration of a therapeutic biodegradable support of the type described herein to an organism confers a beneficial effect on the recipient in terms of amelioration of the undesirable condition. Herein “therapeutic amounts” refers to the amount of the therapeutic agent and/or the therapeutic biodegradable support necessary to elicit a beneficial effect. Additionally or alternatively, the therapeutic biodegradable support described herein may be used prophylactically for reducing the potential onset or reoccurrence of an undesirable condition in a recipient not currently experiencing an undesirable condition.

For example, in some embodiments, the patient may be characterized as having been diagnosed with, experiencing symptoms associated with, wishing to prevent, or otherwise wishing to treat, medicate, correct, lessen the symptoms associated with, a disease, an illness, condition, an abnormality, a predisposition, and/or one or more of the symptoms associated therewith.

Additionally or alternatively, in some embodiments, the patient may be characterized as having recently undergone or as planning to undergo a corrective procedure, a restorative procedure, an elective procedure, a cosmetic procedure, an urgent procedure, a life-extending procedure, a life-saving procedure, or combinations thereof, any of which may be undertaken or supplemental to a treatment of the type described herein. For example, in some embodiments a therapeutic biodegradable support may be employed in ophthalmologic surgery, middle ear surgery, endoscopic sinus surgery, a surgery to improve airway collapse in obstructive sleep apnea, vocal cord surgery, tracheobronchial surgical procedures, gastrointestinal procedures or surgery, urologic surgery, gynecologic surgery for the enhancement or prevention of the likelihood of pregnancy, neurosurgery such as vertebral corrective procedures, endovascular surgery, cosmetic surgery, reconstructive surgery, a surgery to repair traumatic or burn defects, or combinations thereof.

When deployed, the therapeutic biodegradable support may be configured to perform any suitable therapeutic function or combination of therapeutic functions, particularly, when deployed with respect to the tissue site. For example, the therapeutic biodegradable support may be configured to replace missing biological tissue during tissue regrowth; to maintain damaged biological tissue in a particular orientation or placement; to correct deformed biological tissue; to maintain the patency of a passageway, cavity, or orifice; to constrict a passageway, cavity, or orifice; to inhibit constriction of a passageway, cavity, or orifice; to promote constriction of a passageway, cavity, or orifice; to maintain ventilation or free exchange of fluids into and/or out of a physiologic chamber; to physically impede access to a physiologic chamber; or combinations thereof. In some embodiments, the therapeutic biodegradable support may be configured to apply a force with respect to a tissue, for example, a force sufficient to displace the tissue toward a desired position and/or a force sufficient to maintain the tissue at a desired position.

In some embodiments, a method of utilizing or using a therapeutic biodegradable support may comprise administering the therapeutic biodegradable support to a patient in need thereof to treat an undesirable condition of the type(s) disclosed herein.

In some embodiments, a therapeutic biodegradable support may be deployed by a physician in a clinical setting. For example, in some embodiments a therapeutic biodegradable support may be deployed by the physician at, within, about, or otherwise proximate to an intended site of deployment within the patient where an undesirable condition exists. In various embodiments, the site of deployment may include a bodily cavity, a lumen, a vessel, or duct. For example, the site of deployment may include at least a portion of the cranial cavity; at least a portion of the spinal cavity; at least a portion of the thoracic cavity; at least a portion of the abdominal cavity; at least a portion of the pelvic cavity; a blood vessel, vein, or artery; a lymphatic vessel, node, or duct; at least a portion of the gastrointestinal tract; at least a portion of the upper respiratory tract, such as the nasal cavity and paranasal sinuses, pharnyx, larynx; at least a portion of the lower respiratory tract, such as the trachea; or at least a portion of the reproductive, urinary, and/or genital tracts, such as the uterus and fallopian tubes

In some embodiments, the therapeutic biodegradable support may be compressible, for example, radially compressible. In such embodiments, the therapeutic biodegradable support may be compressed and/or contracted (e.g., radially compressed) loaded into a suitable deployment tool (e.g., a plunger or syringe-type deployment tool) configured to selectively retain the therapeutic biodegradable support in such a radially compressed conformation. The deployment tool may then be brought into the vicinity of the intended site of deployment (e.g., tissue), extruded or otherwise ejected or emptied from the deployment device, and allowed to radially expand. Alternatively, the therapeutic biodegradable support may be deployed utilizing one or more conventionally-utilized surgical instruments, such as forceps (e.g., bayonet forceps) and/or a speculum (e.g., a nasal speculum). For example, upon providing access to the site of deployment (e.g., with a speculum or other, similar device), the therapeutic biodegradable support may be brought into the vicinity of the intended site of deployment and released. Upon deployment, the therapeutic biodegradable support may contact the intended target tissue, for example, such that the propensity for radial expansion exhibiting by the therapeutic biodegradable support exerts a force so as to maintain patency of the lumen or other passageway.

Additionally or alternatively, in some embodiments, a therapeutic biodegradable support may be deployed by a physician in conjunction with an operative procedure. For example, using a syringe-like or plunger-like type of deployment tool or any suitable mode of deployment, a surgeon may bring the apparatus, previously having been loaded with a therapeutic biodegradable support, into the vicinity of the intended target tissue for treatment, and thus deploy the device, for example, during the course of an operative procedure. In some embodiments, a therapeutic biodegradable support may be deployed post-operatively. For example, in some embodiments, a therapeutic biodegradable support could be deployed into (e.g., allowed to radially expand within) a lumen or passageway, for example, to maintain postoperative patency for a defined period of time while (when a therapeutic agent is present) simultaneously delivering the therapeutic agent to aid in therapy and healing of the surgical site.

Additionally or alternatively, in some embodiments, the therapeutic biodegradable support may be configured to not necessarily expand or contract, but rather provide predictable time-release of a therapeutic agent at an intended target site and/or provide a structural barrier. In some embodiments, a therapeutic biodegradable support may be deployed within a lumen or other passageway. For example, a therapeutic biodegradable support may be deployed within a lumen or other passageway for the purpose of providing and/or maintaining patency of such lumen or passageway. In such an embodiment, the therapeutic biodegradable support may be deployed within the external auditory canal, the Eustachian tube, the nasolacrimal system, the upper or lower airway, the esophagus, the gastrointestinal tract, the hepatobiliary tract, the pancreatic duct, the genitourinary tract including the male and female reproductive tracts, intravascularly, inter-vertebrally, within the spinal canal or nerve foramena, at the anastamotic site of a vascular repair such as in a vascular bypass procedure or a microvascular free flap tissue transfer procedure, or in combinations thereof.

Additionally or alternatively, in some embodiments, a therapeutic biodegradable support may be deployed around a tissue or other anatomical structure. For example, a therapeutic biodegradable support may be deployed around a tissue or other anatomical structure for example, for the purpose of delivering one or more therapeutic agents to the anatomical structure, or in the case of a vascular structure, to the anatomical structures downstream from it, for the purpose of occluding partially or completely this tissue, or combinations thereof. For example, in an embodiment, a deployment apparatus could be used by a surgeon to narrow the lower esophagus or the lower ureter in undesirable conditions such as gastroesophageal reflux disease or vesicoureteral reflux disease, respectively. In another embodiment, such a therapeutic biodegradable support could be used for complete, permanent occlusion of an anatomical structure, such as the Fallopian tube, to prevent an egg from reaching the uterus for possible fertilization, in the vas deferens for prevention of sperm reaching the eventual semen and ejaculate, or in the feeding vessel of an aneurysm or varicosity to completely prevent vascular inflow.

In some embodiments, the therapeutic biodegradable support may be employed as a component of a therapeutic system. The therapeutic system may further comprise one or more other components generally configured for use with the therapeutic biodegradable support, for example, in order to improve the efficacy the therapeutic biodegradable support and/or to modify the characteristics of the therapeutic biodegradable support in a therapeutic environment.

In some embodiments, the therapeutic system may further comprise an irrigation kit including an irrigation solution, for example, which may be effective to modify the degradation characteristics of the therapeutic biodegradable support. In some embodiments, the irrigation solution may comprise water (H20, e.g., distilled water or deionized water), a salt, and hydrogen peroxide (H202). The water and hydrogen peroxide may be present in a ratio of from about 150:1 to about 250:1, or from about 180:1 to about 220:1. The salt, for example, sodium chloride (NaCl), sodium bicarbonate (NaHCQ3), or combinations thereof, may be present in a quantity sufficient to render the irrigation solution substantially isotonic, alternatively, hypertonic, alternatively, hypotonic. In some embodiments, the irrigation solution may be characterized as acidic, for example, as having a pH that is substantially the same or less than the pH of the tissue site at which the therapeutic biodegradable support is deployed. Not intending to be bound by theory, the hydrogen peroxide present within the irrigation solution may lower pH at tissue site, which may be effective to improve degradation of the therapeutic biodegradable support. Additionally or alternatively, in some embodiments, an irrigation solution may comprise another suitable pH-modifying agent, for example, a weak acid.

In embodiments where the therapeutic biodegradable support is employed as a component of a therapeutic system that also includes an irrigation solution, the irrigation solution may be used to irrigate the therapeutic biodegradable support after deployment with respect to the tissue site intended to receive therapy. For example, the irrigation solution may be used to irrigate the therapeutic biodegradable support at various interval, for example, daily, once every two days, once every three days, once every four days, once every five days, once every six days, once every week, once every ten days, or at any desired interval. Not intending to be bound by theory, irrigation solution may be effective to modify the degradation characteristics associated with the therapeutic biodegradable support.

### Particular Embodiments

One or more embodiments of the therapeutic biodegradable support and methods of utilizing the same having been generally disclosed, the following particularized embodiments of the therapeutic biodegradable support and methods of using such a therapeutic biodegradable support are also disclosed. Although the following embodiments may set forth particular embodiments of the therapeutic biodegradable support and/or particular methods of utilizing that particular therapeutic biodegradable support, this disclosure should not be construed as limiting the disclosed embodiments of a therapeutic biodegradable support to any particular method of utilization, nor should any particular method of utilizing a therapeutic biodegradable support be limited to any particular therapeutic biodegradable support.

### Nasal Splint Embodiments

In some embodiments, the therapeutic biodegradable support comprises and/or is configured to be effective as a nasal splint. For example, in some embodiments, a therapeutic biodegradable support comprising a nasal splint may be configured for placement within and/or at least partially within the nasal cavity, for example, to stabilize a tissue site. In some embodiments the nasal splint may be configured to stabilize a surgically-straightened nasal septum and/or an out-fractured inferior turbinate. Additionally or alternatively, in some embodiments, the nasal splint may be configured to apply a mechanical force to a portion of a tissue site, as an alternative to a surgical procedure or in additional to a surgical procedure, so as to displace some part of the tissue site. For example, the nasal splint may displace some part of the tissue site so as to thereby straighten a nasal septum and/or to outwardly displace an inferior turbinate.

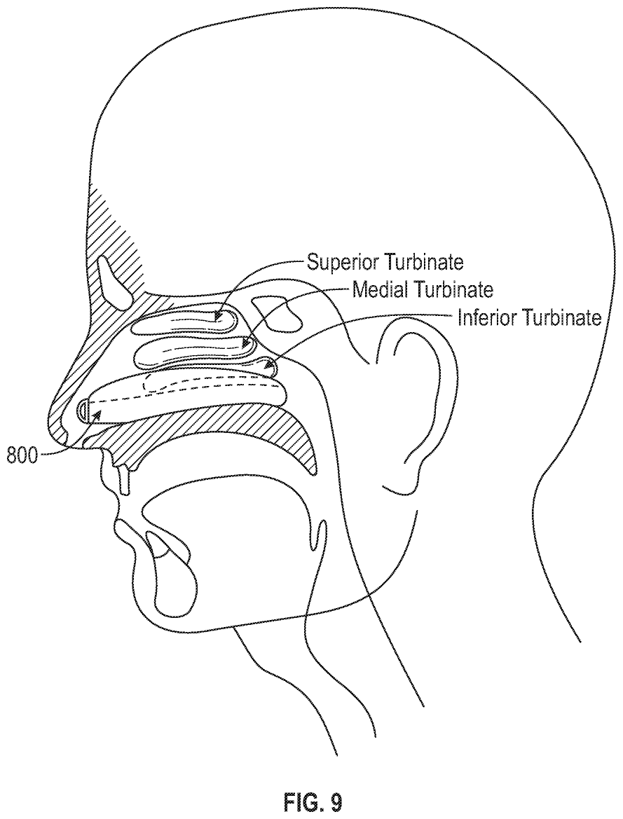

Referring to FIG. 8, a first embodiment of a therapeutic biodegradable support comprising a nasal splint 800 is illustrated. In the embodiment of FIG. 8, the nasal splint is illustrated as deployed within a nasal passage and may be characterized as, for example, generally similar in form and/or structure to a Doyle-type nasal splint. In the embodiment of FIG. 8, the nasal splint 800 generally comprises a hollow, cylindrical (e.g., a tube-like) component 820 and a flap 810. In various embodiments, and as will be appreciated by one of skill in the art upon viewing this disclosure, the nasal splint may be configured such that the nasal splint 800 may be deployed in either the left or the right nasal cavity and may be sized accordingly, for example, having a diameter and/or a length sized for placement within the nasal cavity. For example, referring to FIG. 9, the nasal splint 800 is illustrated deployed within the nasal cavity of a patient. In the embodiment of FIG. 9, the nasal splint 800 is positioned substantially adjacent to the nasal septum and the anterior and medial edges of the inferior turbinate. Additionally, in the embodiment of FIG. 9, the nasal splint 800 is deployed such that the flap 810 abuts the nasal septum and such that the hollow, cylindrical component 820 abuts the medial surface of the inferior turbinate. In some embodiments, the nasal splint 800 may generally rest along the floor of the nasal cavity.

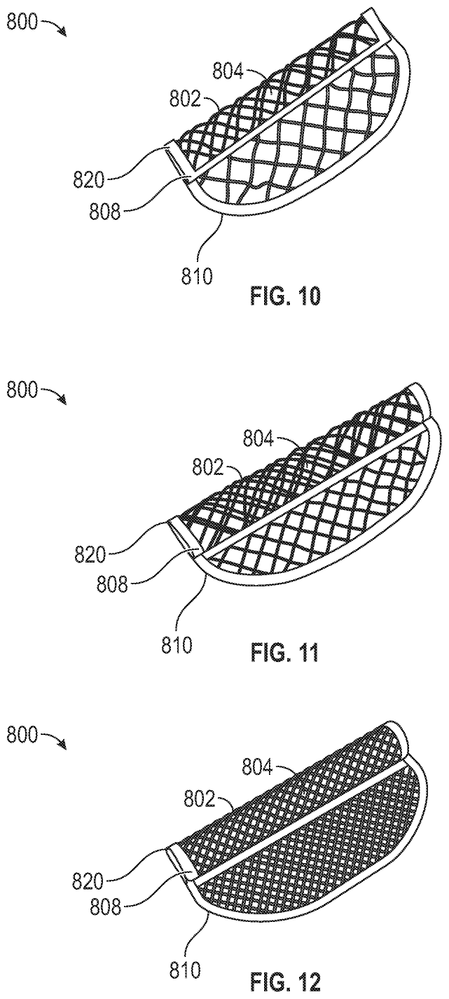

Referring to FIGS. 10, 11, and 12, various embodiments of the nasal splint 800, for example, which may be suitably employed in the context illustrated in FIGS. 8 and 9, are illustrated.

In the embodiments of FIGS. 10, 11, and 12, the nasal splint 800 may be formed from a substantially flat sheet that is formed into the flap 810 and the cylindrical component 820. For example, the sheet, or a portion thereof, may be rolled or folded to yield the cylindrical component 820 and the remainder of the sheet may form the flap 810. In some embodiments, the sheet may comprise a shoulder 830 or other abutment against which the edge of the portion of the sheet that is rolled or folded may be engaged, for example, so as to hold the rolled portion of the sheet in the rolled conformation when the portion of the structural component of the nasal splint is rolled or folded. In some embodiments, the shoulder 830 may generally comprise an area of increased thickness generally extending parallel to the cylindrical component 820.

Also in the embodiments of FIGS. 10, 11, and 12, the flap 810 and the cylindrical component 820 each comprise or are formed from a plurality of strands 802 that intersect to form a mesh or network, for example, generally having diamond-shaped open-spaces 804 there-between (e.g., a waffle-shaped pattern). In various embodiments, the sizing of the strands 802 and spacing of the strands 802 may be varied to achieve a desired infill percentage, which herein refers to the portion of the two-dimensional (2D) area within a bounded space that is filled (as opposed to the portion of the two-dimensional (2D) that remains open). In various embodiments, the nasal splint 800 of the type illustrated in FIGS. 10, 11, and 12 may be characterized as having an infill percentage, for example, the percent of space occupied by the structure of the nasal splint, from about 10% of the two-dimensional area to about 60% of the two-dimensional area, or from about 10% to about 50%, or about 10%, or about 15%, or about 20%, or about 25%, or about 30%, or about 35%, or about 40%. For example, the embodiment of FIG. 10 illustrates an embodiment having about 10% infill; the embodiment of FIG. 11 illustrates an embodiment having about 20% infill; and the embodiment of FIG. 12 illustrates an embodiment having about 30% infill.

For example, in the embodiments of FIGS. 10, 11, and 12, each of the plurality of strands 802 is joined at an end or terminus thereof to multiple other strands 802. As such, the plurality of strands 802 together form the structure of the nasal splint 800. In the embodiments of FIGS. 10, 11, and 12, the open-spaces 804 are illustrated as generally diamond-shaped, although in other embodiments the open-spaces 804 may be square, rectangular, triangular, circular, ovoid, oblong, or irregularly-shaped. The open-spaces 804 may collectively define the percent of space not occupied by the structure of the nasal splint. For example, a nasal splint having an infill of about 20% would, correspondingly, have open-spaces 804 occupying about 80% of the two-dimensional area of the nasal splint 800. Also, in some embodiments the nasal splint 800 may comprise a perimeter component 808 generally disposed around the edges of the nasal splint 800, for example, joined to a peripheral portion of the strands 802. In some embodiments, the perimeter component 808 may have a width and/or thickness that is wider and/or thicker than the strands 802 of the nasal splint 800.

In some embodiments, a nasal splint 800 formed from a plurality of intersecting strands 802 to form a mesh or network, for example, the nasal splint 800 illustrated in FIGS. 10, 11, and 12, may be advantageously employed within the nasal cavity, such as illustrated with respect to FIGS. 8 and 9. For example, the nasal splint 800 formed from or comprising such a plurality of intersecting strands 802, thereby forming corresponding open-spaces 804 between the various respective strands 802, may allow improved air-flow through the nasal cavity and, as such, may also improve the ease with which the patient is able to breath while the nasal splint 800 is deployed within their nasal cavity. Additionally or alternatively, the nasal splint 800 comprising such a plurality of intersecting strands 802, thereby forming corresponding open-spaces 804 between the various respective strands 802, may impart improved degradability to the nasal splint 800 when deployed. Not intending to be bound by theory, the open-spaces 804 between the strands 802 may allow improved contact between the plurality of strands 802 and the fluid (such as air, an irrigation solution, or mucosa of the patient), which may have the effect of causing the strands 802 to degrade more consistently over time.

Referring to FIG. 13, another embodiment of the nasal splint 800 is illustrated. In the embodiment of FIG. 13, the flap 810 and the cylindrical component 820 each comprise or are formed from a plurality of strands or struts 806, extending generally perpendicularly to the longitudinal axis of the cylindrical component 820. In the embodiment of FIG. 13, the plurality of struts 806 may are joined together to form network, for example, generally having diamond-shaped open-spaces 804 there-between In the embodiment of FIG. 13, the nasal splint 800 also comprises a perimeter component 808 generally disposed around the edges of the nasal splint 800, for example, joined to a peripheral portion of the strands.

In various embodiments, the nasal splint 800 of the type illustrated in FIG. 13 may be characterized as having an infill percentage from about 10% of the two-dimensional area to about 40% of the two-dimensional area, or from about 10% to about 50%, or about 10%, or about 15%, or about 20%, or about 25%, or about 30%. For example, FIG. 13 illustrates an embodiment having about 20% infill.

In some embodiments, the nasal splint 800 may be configured to exert a force, for example, an outward force, upon deployment within the nasal cavity so as to stabilize (e.g., medialize) a surgically-corrected (e.g., surgically-straightened) nasal septum and/or to lateralize the inferior turbinate (e.g., to exert a lateralizing force against the inferior turbinate), for example, after outfracture, cauterization, radiofrequency ablation, submucous resection, the like, or combinations thereof. For example, the nasal splint may be configured to provide such a radially outward force to provide septal support, and/or to reduce or prevent stenosis of the nasal cavity. For example, in the embodiment of FIGS. 8 and 9, the nasal splint 800 may be radially compressed for placement within the nasal cavity (e.g., the flap may be rolled over and/or partially around the hollow cylinder and, optionally, held in place with one or more biodegradable bands such as biodegradable, elastomeric bands). The nasal splint 800 may exert a force radially outward, for example, so as to apply a physical force to the nasal septum and/or the inferior turbinate upon placement and deployment within the nasal cavity. For example, the nasal splint 800 may exhibit mechanical activity so as to exert a force against the inferior turbinate effective to cause at least a portion of the inferior turbinate to be displaced laterally and/or to the side, and thereby increase the diameter of the airway.

In some embodiments, a method of utilizing a therapeutic biodegradable support comprising a nasal splint may comprise deploying the therapeutic biodegradable support comprising the nasal splint within the nasal cavity of a patient. In various embodiment, such a patient may be characterized as having recently undergone a septoplasty and/or an inferior turbinate reduction, as suffering from (e.g., experiencing one or more signs, symptoms, or the like, associated with) allergic rhinitis, viral rhinitis, rhinosinusitis of viral or bacterial or unknown origin, and/or vasomotor (non-allergic) rhinitis, as suffering from an external and/or internal nasal valve collapse (e.g., as may result from exertion during inspiration, such as during exercise), as suffering from snoring and/or obstructive sleep apnea (e.g., as may result from an external or internal nasal valve collapse), or combinations thereof.

In an embodiment, the nasal splint 800 comprises a degradable composition comprises a degradable polymer, particularly, a copolymer, comprising glycolide, trimethyl carbonate, and caprolactone subunits. For example, the degradable polymer may comprise a copolymer comprising from about 50% to about 60% glycolide subunits, from about 20% to about 30 trimethyl carbonate subunits, and from about 10% to about 30% caprolactone subunits, on the basis of the number of subunits present within the copolymer. In such an embodiment, the nasal splint may be formed from a plurality of strands that intersect to form a mesh or network, for example, as illustrated in FIGS. 10, 11, and 12. The nasal splint may further be characterized as exhibiting a resistance to compression of about 0.5 kg and a degradation profile such that at least about 30% of the nasal splint is degraded within a duration of time, commencing from deployment in a biological environment, of from 7 days to about 35 days, or from about 14 days to about 28 days.

In another embodiment, the nasal splint 800 comprises a degradable composition comprises a first degradable polymer, particularly, a copolymer, comprising glycolide, trimethyl carbonate, and caprolactone subunits. For example, the degradable composition may comprise from about 70% to about 99.99% by weight of the first polymer and from about 0.01% to about 30% of the second polymer, by weight of the degradable composition. The first degradable polymer may comprise a copolymer comprising from about 50% to about 60% glycolide subunits, from about 20% to about 30 trimethyl carbonate subunits, and from about 10% to about 30% caprolactone subunits. The second degradable polymer may comprise chitosan being from about 8% to about 20% acetylated, for example, for about 12% to about 18% acetylated, for example, about 16% acetylated. In such an embodiment, the nasal splint 800 may be formed from a plurality of strands 802 that intersect to form a mesh or network, for example, as illustrated in FIGS. 10, 11, and 12. The nasal splint may further be characterized as exhibiting a resistance to compression of about 0.5 kg and a degradation profile such that at least about 30% of the nasal splint is degraded within a duration of time, commencing from deployment in a biological environment, of from 7 days to about 35 days, or from about 14 days to about 28 days.

In another embodiment, the nasal splint 800 comprises a degradable composition comprising at least about 95% chitosan, or at least about 97% chitosan, or at least about 98% chitosan, or at least about 99% chitosan, or at least about 99.5% chitosan by weight of the degradable composition. The chitosan may be from about 8% to about 20% acetylated, for example, for about 12% to about 18% acetylated, for example, about 16% acetylated. In such an embodiment, the nasal splint may be formed from a plurality of strands that intersect to form a mesh or network, for example, as illustrated in FIGS. 10, 11, and 12. The nasal splint may further be characterized as exhibiting a resistance to compression of about 0.5 kg and a degradation profile such that at least about 30% of the nasal splint is degraded within a duration of time, commencing from deployment in a biological environment, of from 7 days to about 35 days, or from about 14 days to about 28 days.

In an embodiment, the nasal splint 800 may comprise a suitable therapeutic agent. In some embodiments, such as one or more of the embodiments previously disclosed herein, the therapeutic agent may be included and/or incorporated within the degradable composition. Additionally or alternatively, the therapeutic agent may be applied to one or more surfaces of the nasal splint 800, for example, by spraying or dipping the nasal splint 800 using a mixture including the therapeutic agent dispersed in a carrier. When applied to one or more surfaces of the nasal splint 800, the chitosan may be applied continuously or discontinuously and may be applied in any suitable thickness, for example, from about 1 micron to about 100 microns.

In some embodiments, the therapeutic agent comprises chitosan. For example, the nasal splint may include from about 0.01% to about 50% chitosan, by weight of the degradable material. The chitosan may be from about from about 8% to about 20% acetylated, for example, for about 12% to about 18% acetylated, for example, about 16% acetylated % acetylated. For example, in various embodiments, the degradable material may comprise a copolymer comprising from about 50% to about 60% glycolide subunits, from about 20% to about 30 trimethyl carbonate subunits, and from about 10% to about 30% caprolactone units, a degradable polymer such as chitosan, or combinations thereof.

Additionally or alternatively, in some embodiments, the nasal splint 800 comprises a suitable API or combination of APis, dependent upon the patient and/or the conditions experienced by the patient.

In some embodiments, the API may comprise a corticosteroid such as beclomethasone, budesonide, ciclesonide, fluticasone furoate, fluticasone propionate, flunisolide, mometasone, or triamcinolone, an antihistamine such as azelastine or olopatadine, an anticholinergic agent such as ipratroprium bromide, an antibiotic such as, but not limited to, doxycycline, mupirocin, trimethoprim/sulfamethoxasole or combinations thereof. In some embodiments where the patient is characterized as suffering from allergic rhinitis, viral rhinitis, rhinosinusitis of viral or bacterial or unknown origin, and/or vasomotor (non-allergic) rhinitis, the API may comprise a corticosteroid such as beclomethasone, budesonide, ciclesonide, fluticasone furoate, fluticasone propionate, flunisolide, mometasone, or triamcinolone, a topical antihistamine such as azelastine or olopatadine, an anticholinergic agent such as ipratroprium bromide, an antibiotic (such as, but not limited to, muplrocm, doxycycline, trimethoprim/sulfamethoxasole, levofloxacin) or combinations thereof. In some embodiment where the patient is characterized as suffering from an external and/or internal nasal valve collapse (e.g., as may result from exertion during inspiration, such as during exercise), the API may comprise an antibiotic such as, but not limited to, mupirocin, doxycycline, trimethoprim/sulfamethoxasole, levofloxacin, or combinations thereof. In some embodiments where the patient is characterized as suffering from snoring and/or obstructive sleep apnea (e.g., as may result from an external or internal nasal valve collapse), the API comprises a corticosteroid such as beclomethasone, budesonide, ciclesonide, fluticasone furoate, fluticasone propionate, flunisolide, mometasone, or triamcinolone, a topical antihistamine such as azelastine or olopatadine, an anticholinergic agent such as ipratroprium bromide, and antibiotic such as, but not limited to, mupirocin, or combinations thereof. When present, the API may confer a therapeutic benefit upon the tissue at the site of deployment (e.g., the operative site).

In an embodiment, the nasal splint 800 may be deployed by a physician. For example, the nasal splint 800 may be deployed by utilization of a suitable plunger or syringe-type deployment tool, alternatively, via any suitable mode of deployment. In some embodiments, the nasal splint 800 will be compressible, for example, radially compressible. In such an embodiment, the nasal splint 800 may be compressed and/or contracted (e.g., radially compressed) loaded into the plunger or syringe-type deployment tool. The deployment tool embodiment may then be brought into the vicinity of the intended site of deployment (e.g., tissue), extruded or ejected from the deployment device, and allowed to radially expand, for example, thereby allowing for contact with the intended target tissue. Alternatively, the therapeutic biodegradable support may be deployed utilizing conventionally-utilized surgical instruments.

In some embodiments, the nasal splint 800 may be characterized as exhibiting at least about 95% degradation in a duration of from about 5 days to about 60 days, or from about 7 days to about 30 days, or about 7 to 10 days, or about 28 to 31 days. In some embodiments, the nasal splint 800 may be characterized as exhibiting at least about 95% elution of the API in a duration of about 5 days to about 45 days, or from about 7 days to about 30 days, or about 7 to 10 days, or about 28 to 31 days.

In some embodiments, a therapeutic biodegradable support comprising a nasal splint may be configured for placement within or at least partially within the paranasal sinuses, for example, within or at least partially within the ethmoid sinuses and/or within or at least partially within the sphenoid, frontal, and/or maxillary sinuses. In some embodiments the nasal splint may be configured to maintain patency of the ethmoid sinuses. For example, the nasal splint may be employed as an adjunct to an ethmoidectomy. In an ethmoidectomy, portions of tissue defining the individual ethmoid sinuses may be removed. The nasal splint may be employed to maintain the patency of the ethmoid sinuses during healing, for example, such that upon healing, the ethmoid sinuses exhibit improved patency. In some embodiments, for example, where the nasal splint includes a therapeutic agent such as chitosan, the nasal splint may be employed to provide hemostasis, antimicrobial (e.g., antibacterial, anti-fungal, and anti-viral) activity to the surgical site.

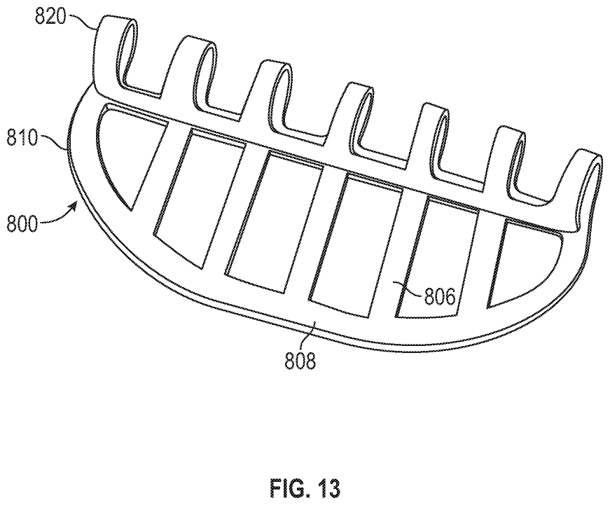

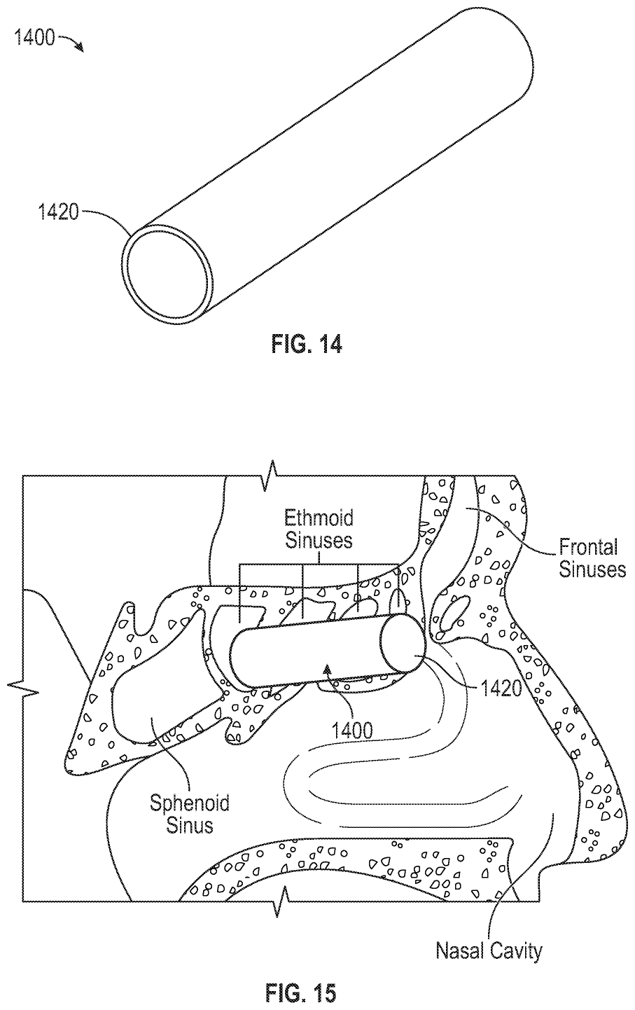

Referring to FIGS. 14 and 15, an embodiment of a therapeutic biodegradable support comprising a nasal splint 1400 is illustrated. In the embodiment of FIG. 15, the nasal splint is illustrated as deployed within an ethmoid sinus. In the embodiment of FIGS. 14 and 15, the nasal splint 1400 generally comprises a hollow, cylindrical (e.g., a tube-like) component 1420. In various embodiments, and as will be appreciated by one of skill in the art upon viewing this disclosure, the nasal splint may be configured such that the nasal splint 1400 may be deployed in either the left or the right ethmoid sinus and may be sized accordingly, for example, having a diameter and/or a length sized for placement within the ethmoid sinus.

In some embodiments, a method of utilizing a therapeutic biodegradable support comprising a nasal splint may comprise deploying the therapeutic biodegradable support comprising the nasal splint within the paranasal sinuses, for example, within the ethmoid sinuses, of a patient. In various embodiment, such a patient may be characterized as having recently undergone a ethmoidectomy, as suffering from (e.g., experiencing one or more signs, symptoms, or the like, associated with) allergic rhinitis, viral rhinitis, rhinosinusitis of viral or bacterial or unknown origin, and/or vasomotor (non-allergic) rhinitis, and/or obstructive sleep apnea (e.g., as may result from an external or internal nasal valve collapse), or combinations thereof.

In some embodiments, the nasal splint 1400 may comprise a suitable therapeutic agent. In some embodiments, the therapeutic agent comprises chitosan. In the embodiment of FIGS. 14 and 15, the nasal splint 1400 comprises a degradable composition comprising at least about 95% chitosan, or at least about 97% chitosan, or at least about 98% chitosan, or at least about 99% chitosan, or at least about 99.5% chitosan by weight of the degradable composition. The chitosan may be from about 8% to about 20% acetylated, for example, for about 12% to about 18% acetylated, for example, about 16% acetylated.

Additionally or alternatively, in some embodiments, the nasal splint 1400 comprises a suitable API or combination of APis, dependent upon the patient and/or the conditions experienced by the patient.

In some embodiments, the API may comprise a corticosteroid such as beclomethasone, budesonide, ciclesonide, fluticasone furoate, fluticasone propionate, flunisolide, mometasone, or triamcinolone, an antihistamine such as azelastine or olopatadine, an anticholinergic agent such as ipratroprium bromide, an antibiotic such as, but not limited to, doxycycline, mupirocin, trimethoprim/sulfamethoxasole or combinations thereof. In some embodiments where the patient is characterized as suffering from allergic rhinitis, viral rhinitis, rhinosinusitis of viral or bacterial or unknown origin, and/or vasomotor (non-allergic) rhinitis, the API may comprise a corticosteroid such as beclomethasone, budesonide, ciclesonide, fluticasone furoate, fluticasone propionate, flunisolide, mometasone, or triamcinolone, a topical antihistamine such as azelastine or olopatadine, an anticholinergic agent such as ipratroprium bromide, an antibiotic (such as, but not limited to, muplrocm, doxycycline, trimethoprim/sulfamethoxasole, levofloxacin) or combinations thereof. In some embodiment where the patient is characterized as suffering from an external and/or internal nasal valve collapse (e.g., as may result from exertion during inspiration, such as during exercise), the API may comprise an antibiotic such as, but not limited to, mupirocin, doxycycline, trimethoprim/sulfamethoxasole, levofloxacin, or combinations thereof. In some embodiments where the patient is characterized as suffering from snoring and/or obstructive sleep apnea (e.g., as may result from an external or internal nasal valve collapse), the API comprises a corticosteroid such as beclomethasone, budesonide, ciclesonide, fluticasone furoate, fluticasone propionate, flunisolide, mometasone, or triamcinolone, a topical antihistamine such as azelastine or olopatadine, an anticholinergic agent such as ipratroprium bromide, and antibiotic such as, but not limited to, mupirocin, or combinations thereof. When present, the API may confer a therapeutic benefit upon the tissue at the site of deployment (e.g., the operative site).

In an embodiment, the nasal splint 1400 may be deployed by a physician. For example, the nasal splint 1400 may be deployed by utilization of a suitable plunger or syringe-type deployment tool, alternatively, via any suitable mode of deployment. In some embodiments, the nasal splint 1400 will be compressible, for example, radially compressible. In such an embodiment, the nasal splint 1400 may be compressed and/or contracted (e.g., radially compressed) loaded into the plunger or syringe-type deployment tool. The deployment tool embodiment may then be brought into the vicinity of the intended site of deployment (e.g., tissue), extruded or ejected from the deployment device, and allowed to radially expand, for example, thereby allowing for contact with the intended target tissue. Alternatively, the therapeutic biodegradable support may be deployed utilizing conventionally-utilized surgical instruments.

In some embodiments, the nasal splint 1400 may be characterized as exhibiting at least about 30% degradation in a duration of from about 5 days to about 60 days, or from about 7 days to about 30 days, or about 7 to 10 days, or about 28 to 31 days. In some embodiments, the nasal splint 800 may be characterized as exhibiting at least about 95% elution of the API in a duration of about 5 days to about 45 days, or from about 7 days to about 30 days, or about 7 to 10 days, or about 28 to 31 days.

As may be appreciated by one of skill in the art upon viewing this disclosure, nasal splints are used frequently, often in a postoperative setting. A therapeutic biodegradable support comprising a nasal splint, for example, as disclosed herein, offers significant advances over the existing art. For example, because the therapeutic biodegradable support degrades/biodegrades, uncomfortable removal procedures are avoided. Also, by delivering therapeutic agents at the surgical site, which current devices do not allow for, the therapeutic biodegradable support, when utilized postoperatively, may dramatically speed up healing and minimize scarring, as well as, lessen or eliminate the costs and risks of systemic side effects associated with an oral prescription antibiotic and/or corticosteroid. Further, a therapeutic biodegradable support comprising a nasal splint may provide significant therapeutic benefit. For example, such a therapeutic biodegradable support may allow for targeted delivery of a therapeutic agent (e.g., chitosan or an API) into the nasal cavity. In such embodiments, the targeted delivery of such a therapeutic agent may yield a substantial decrease in any obstructions within the nasal passage, for example, by decreasing the size of the inferior turbinate. The therapeutic agent may also prevent scar formation, and/or reduce the likelihood of revisionary surgery.

One of skill in the art, upon viewing this disclosure, will appreciate one or more additional embodiments and/or variations of a therapeutic biodegradable support as disclosed herein and/or methods of utilizing the same. As such, the forgoing is in no way intended to be limited to the embodiments or example disclosed herein.

At least one embodiment is disclosed and variations, combinations, and/or modifications of the embodiment(s) and/or features of the embodiment(s) made by a person having ordinary skill in the art are within the scope of the disclosure. Alternative embodiments that result from combining, integrating, and/or omitting features of the embodiment(s) are also within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, RI, and an upper limit, Ru, is disclosed, any number falling within the range is specifically disclosed. In particular, the following numbers within the range are specifically disclosed: R=Rl+k*(Ru−Rl), wherein k is a variable ranging from 1 percent to 100 percent with a 1 percent increment, i.e., k is 1 percent, 2 percent, 3 percent, 4 percent, 5 percent, . . . , 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of the term “optionally” with respect to any element of a claim means that the element is required, or alternatively, the element is not required, both alternatives being within the scope of the claim. Use of broader terms such as comprises, includes, and having should be understood to provide support for narrower terms such as consisting of, consisting essentially of, and comprised substantially of. Accordingly, the scope of protection is not limited by the description set out above but is defined by the claims that follow, that scope including all equivalents of the subject matter of the claims. Each and every claim is incorporated as further disclosure into the specification and the claims are embodiment(s) of the present invention

1. A biodegradable nasal splint comprising:
a tubular component at least partially defining a hollow passageway, wherein the tubular component is formed from a degradable material comprising a copolymer comprising glycolide subunits, trimethyl carbonate subunits, and caprolactone subunits.

2. The biodegradable nasal splint of claim 1, wherein the biodegradable splint comprises at least 90% by weight of one or more degradable materials.

3. The biodegradable nasal splint of claim 1, wherein the degradable material is configured such that at least 95% by weight of the degradable material will degrade in a duration of from about 7 days to about 30 days upon deployment within a nasal passage.

4. The biodegradable nasal splint of claim 1, wherein the degradable material further comprises from about 0.01% to about 30% chitosan, by weight of the degradable material.

5. The biodegradable nasal splint of claim 1, wherein the biodegradable nasal splint further comprises a therapeutic agent applied to one or more surfaces of the nasal splint, wherein the therapeutic agent comprises chitosan.

6. The biodegradable nasal splint of claim 1, further comprising an therapeutic agent comprising a corticosteroid, an antihistamine, an anticholinergic agent, an antibiotic, or combinations thereof.

7. The biodegradable nasal splint of claim 6,
wherein the corticosteroid comprises beclomethasone, budesonide, ciclesonide, fluticasone furoate, fluticasone propionate, flunisolide, mometasone, triamcinolone, or combinations thereof,
wherein the antihistamine comprises azelastine, olopatadine, loratadine, desloratadine, fexofenadine, cetirizine, levocetirizine, or combinations thereof, wherein the anticholinergic agent comprises ipratroprium bromide, meclozine, or combinations thereof,
wherein the antibiotic comprises mupirocin, doxycycline, trimethoprim/sulfamethoxasole, levofloxacin, ciprofloxacin, cephalexin, amoxicillin, clavulanic acid or combinations thereof,
wherein the antiviral comprises acyclovir, valcyclovir, or combinations thereof, or
wherein the antifungal comprises an allylamine, an imidazole, a polyenes, a thiocarbamate, a triazole, a derivative thereof, or combinations thereof.

8. The biodegradable nasal splint of claim 1, wherein the biodegradable nasal splint is configured for placement between a septum and an inferior turbinate so as to apply a horizontally opposing force between the nasal septum and the inferior turbinate.

9. A method comprising:
performing a corrective procedure with respect to a patient's nasal passage, wherein the corrective procedure comprises adjusting or removing at least a portion of a nasal septum of the patient or lateralizing an inferior turbinate of the patient;
positioning biodegradable nasal splint comprising a tubular component at least partially defining a hollow passageway within the patient's nasal passage between the nasal septum and an inferior turbinate, wherein the tubular component is formed from a degradable material comprising a copolymer comprising glycolide subunits, trimethyl carbonate subunits, and caprolactone subunits.

10. The method of claim 9, wherein the degradable material further comprises from about 0.01% to about 30% chitosan, by weight of the degradable material.

11. The method of claim 9, further comprising allowing at least a portion of the biodegradable nasal splint to degrade within the patient's nasal passage.

12. The method of claim 9, wherein the biodegradable nasal splint further comprises a therapeutic agent applied to one or more surfaces of the nasal splint, wherein the therapeutic agent comprises chitosan.

13. The method of claim 9, wherein the patient is characterized as experiencing allergic rhinitis, viral rhinitis, influenza, rhinosinusitis of a viral, a bacterial, or unknown origin, vasomotor rhinitis, or combinations thereof.

14. The method of claim 9, wherein the patient is characterized as experiencing an external and/or internal nasal valve collapse, snoring and/or obstructive sleep apnea, or combinations thereof.

15. The method of claim 9, wherein the biodegradable splint comprises at least 90% by weight of the degradable material.

16. The method of claim 9, wherein the biodegradable nasal splint further comprises an active ingredient comprising a corticosteroid, an antihistamine, an anticholinergic agent, an antibiotic, an antiviral, an antifungal, or combinations thereof.

17-27. (canceled)
